SEQUENCE LISTING (282 sequences)

SEQ ID NO: 1 - DNA, 21 bp, Homo sapiens
aatatctcctccccattctgg

SEQ ID NO: 2 - DNA, 21 bp, Homo sapiens
tgtggttgagaatgagcatgt

SEQ ID NO: 3 - DNA, 42 bp, Homo sapiens
tgtggttgagaatgagcatgtggataccaccttctgtagagt

SEQ ID NO: 4 - DNA, 20 bp, Homo sapiens
atgacacagagctggtagcc

SEQ ID NO: 5 - DNA, 20 bp, Homo sapiens
aaccagaaaatacgagccct

SEQ ID NO: 6 - DNA, 40 bp, Homo sapiens
aaccagaaaatacgagcccttctccatctaccacaggcac

SEQ ID NO: 7 - DNA, 20 bp, Homo sapiens
aaaactgctaaggccaaggt

SEQ ID NO: 8 - DNA, 20 bp, Homo sapiens
tcagcaacctctttcctcac

SEQ ID NO: 9 - DNA, 40 bp, Homo sapiens
tcagcaacctctttcctcactctgattcatctgtgctgcc

SEQ ID NO: 10 - DNA, 20 bp, Homo sapiens
accattgtccagccatcagc

SEQ ID NO: 11 - DNA, 20 bp, Homo sapiens
accctctgctgtccgtgtct

SEQ ID NO: 12 - DNA, 40 bp, Homo sapiens
accctctgctgtccgtgtctctgaaggaggatggagtctg

SEQ ID NO: 13 - DNA, 20 bp, Homo sapiens
ttttaggagaccgaagtccg

SEQ ID NO: 14 - DNA, 20 bp, Homo sapiens
agccaacgtgccatgtgcta

SEQ ID NO: 15 - DNA, 40 bp, Homo sapiens
agccaacgtgccatgtgctacctctgttccttccctctac

SEQ ID NO: 16 - DNA, 20 bp, Homo sapiens
gtaccggcgggcattcagtg

SEQ ID NO: 17 - DNA, 20 bp, Homo sapiens
agagtgagcccagcagaacc

SEQ ID NO: 18 - DNA, 40 bp, Homo sapiens
agagtgagcccagcagaacccgttctcctggatccaaggc

SEQ ID NO: 19 - DNA, 20 bp, Homo sapiens
tacgcagcgcctccctccac

SEQ ID NO: 20 - DNA, 20 bp, Homo sapiens
ctgttctcgtcgtttccgca

SEQ ID NO: 21 - DNA, 40 bp, Homo sapiens
ctgttctcgtcgtttccgcaaccttgggggccttttcatt

SEQ ID NO: 22 - DNA, 20 bp, Homo sapiens
tacgacaaggatagaagcgg

SEQ ID NO: 23 - DNA, 20 bp, Homo sapiens
aggacatgacgctctttctg

SEQ ID NO: 24 - DNA, 42 bp, Homo sapiens
aggacatgacgctctttctggatttcctttttgtttttctcg

SEQ ID NO: 25 - DNA, 20 bp, Homo sapiens
ggatacaaagaagggagtgc

SEQ ID NO: 26 - DNA, 20 bp, Homo sapiens
ccaactcaggacaaggtaca

SEQ ID NO: 27 - DNA, 40 bp, Homo sapiens
ccaactcaggacaaggtacatcttctgggctcttggtggc

SEQ ID NO: 28 - DNA, 20 bp, Homo sapiens
ccagaagaaagcggtcaaga

SEQ ID NO: 29 - DNA, 20 bp, Homo sapiens
aaccttcattttcccctggg

SEQ ID NO: 30 - DNA, 40 bp, Homo sapiens
aaccttcattttcccctgggccagtgatgagcgggttaca

SEQ ID NO: 31 - DNA, 26 bp, Homo sapiens
ggccttgccagagcttttggaatacc

SEQ ID NO: 32 - DNA, 26 bp, Homo sapiens
agccattttcatccaagtttttgaca

SEQ ID NO: 33 - DNA, 52 bp, Homo sapiens
agccattttcatccaagtttttgacagttgattaatttctgaatccccatgg

SEQ ID NO: 34 - DNA, 25 bp, Homo sapiens
ggtgtggacatgtgggctgttggct

SEQ ID NO: 35 - DNA, 25 bp, Homo sapiens
tggtcttggcagctgacatccaggt

SEQ ID NO: 36 - DNA, 45 bp, Homo sapiens
tggtcttggcagctgacatccaggttctagtaagtcgtctcctgc

SEQ ID NO: 37 - DNA, 20 bp, Homo sapiens
tgccggttgtcaaatccctt

SEQ ID NO: 38 - DNA, 20 bp, Homo sapiens
tccgatgcagctcagtaccg

SEQ ID NO: 39 - DNA, 37 bp, Homo sapiens
tccgatgcacagtaccgatgtggattggaacgctgat

SEQ ID NO: 40 - DNA, 30 bp, Homo sapiens
agcagtcttttggagtgaccagcaactttg

SEQ ID NO: 41 - DNA, 30 bp, Homo sapiens
catgcaatgaagctgaacatgaccgtagtt

SEQ ID NO: 42 - DNA, 50 bp, Homo sapiens
catgcaatgaagctgaacatgaccgtagtttctgtgatgagttttaaaaa

SEQ ID NO: 43 - DNA, 18 bp, Homo sapiens
gctgcaggccctgaagga

SEQ ID NO: 44 - DNA, 18 bp, Homo sapiens
ccccgacggtctctcttc

SEQ ID NO: 45 - DNA, 36 bp, Homo sapiens
ccccgacggtctctcttcagttctgagctttcaagg

SEQ ID NO: 46 - DNA, 20 bp, Homo sapiens
tgtggtacaaccacgaacag

SEQ ID NO: 47 - DNA, 20 bp, Homo sapiens
agatatttccgcagcaacag

SEQ ID NO: 48 - DNA, 40 bp, Homo sapiens
agatatttccgcagcaacagatgccacagccaggactaat

SEQ ID NO: 49 - DNA, 19 bp, Homo sapiens
caggagctaaaggcgaaga

SEQ ID NO: 50 - DNA, 19 bp, Homo sapiens
ccaggctgacctcggggac

SEQ ID NO: 51 - DNA, 38 bp, Homo sapiens
ccaggctgacctcggggacgacctccagggacgccatc

SEQ ID NO: 52 - DNA, 21 bp, Homo sapiens
tgaaggtgtggggaagcatta

SEQ ID NO: 53 - DNA, 21 bp, Homo sapiens
ttacaccacaagccaaacgac

SEQ ID NO: 54 - DNA, 42 bp, Homo sapiens
ttacaccacaagccaaacgactgatgcaatggtctcctgaga

SEQ ID NO: 55 - DNA, 24 bp, Homo sapiens
ccaagaggaccaggagaatatcaa

SEQ ID NO: 56 - DNA, 24 bp, Homo sapiens
ggataatcaagagggaccaatggt

SEQ ID NO: 57 - DNA, 44 bp, Homo sapiens
ggataatcaagagggaccaatggtgtgaacgcaggctgtttact

SEQ ID NO: 58 - DNA, 20 bp, Homo sapiens
ggcgcgtctccggcacgatg

SEQ ID NO: 59 - DNA, 25 bp, Homo sapiens
gcagccagcaaaaaagaacagactc

SEQ ID NO: 60 - DNA, 45 bp, Homo sapiens
gcagccagcaaaaaagaacagactcaaagtttcagtgcaagatcc

SEQ ID NO: 61 - DNA, 29 bp, Homo sapiens
tggaattctgttcggtgtttaagccagca

SEQ ID NO: 62 - DNA, 29 bp, Homo sapiens
caatatgggatagcgggtctttaagtcga

SEQ ID NO: 63 - DNA, 58 bp, Homo sapiens
caatatgggatagcgggtctttaagtcgatccaagaggactctcccggaggtttccaa

SEQ ID NO: 64 - DNA, 21 bp, Homo sapiens
catcccccacagcacaacaag

SEQ ID NO: 65 - DNA, 21 bp, Homo sapiens
acagcaggcatgcttcatggt

SEQ ID NO: 66 - DNA, 40 bp, Homo sapiens
acagcaggcatgcttcatgggtctcaccgatacacttccg

SEQ ID NO: 67 - DNA, 21 bp, Homo sapiens
acccccagtctcaatctcaac

SEQ ID NO: 68 - DNA, 21 bp, Homo sapiens
cgttcgggctgaggctggtgc

SEQ ID NO: 69 - DNA, 42 bp, Homo sapiens
cgttcgggctgaggctggtgccgtcaacaggaacccgcaggc

SEQ ID NO: 70 - DNA, 20 bp, Homo sapiens
ggaacttcggaaatccaagg

SEQ ID NO: 71 - DNA, 20 bp, Homo sapiens
ccatgtggagcaggtaggtg

SEQ ID NO: 72 - DNA, 40 bp, Homo sapiens
ccatgtggagcaggtaggtggtgtgccccaggaaagtatt

SEQ ID NO: 73 - DNA, 20 bp, Homo sapiens
ccttcctcctgctggtgtcc

SEQ ID NO: 74 - DNA, 20 bp, Homo sapiens
gccggatgtccttccaggta

SEQ ID NO: 75 - DNA, 43 bp, Homo sapiens
gccggatgtccttccaggtaatcaccaccatgcgctgctgcga

SEQ ID NO: 76 - DNA, 20 bp, Homo sapiens
ggggaagagaagcattgagg

SEQ ID NO: 77 - DNA, 20 bp, Homo sapiens
gcctggtggtcgtggacgct

SEQ ID NO: 78 - DNA, 40 bp, Homo sapiens
gcctggtggtcgtggacgctcgggaatctggggtctagga

SEQ ID NO: 79 - DNA, 20 bp, Homo sapiens
agaaagagaacagcttcgca

SEQ ID NO: 80 - DNA, 20 bp, Homo sapiens
cacattgattcattggctga

SEQ ID NO: 81 - DNA, 40 bp, Homo sapiens
cacattgattcattggctgattttcttccaggattctccc

SEQ ID NO: 82 - DNA, 21 bp, Homo sapiens
gctggatgcccatgagagagg

SEQ ID NO: 83 - DNA, 21 bp, Homo sapiens
catgggaacagctctcgagga

SEQ ID NO: 84 - DNA, 42 bp, Homo sapiens
catgggaacagctctcgaggaatcttgttttctttcatgctc

SEQ ID NO: 85 - DNA, 20 bp, Homo sapiens
ggggacgcagtagccgagat

SEQ ID NO: 86 - DNA, 20 bp, Homo sapiens
tcacttcagcatcacctcca

SEQ ID NO: 87 - DNA, 40 bp, Homo sapiens
tcacttcagcatcacctccataaagggaagagccgagtcg

SEQ ID NO: 88 - DNA, 20 bp, Homo sapiens
cggatgggaatgtcgtttgg

SEQ ID NO: 89 - DNA, 20 bp, Homo sapiens
gggggtctcgcctcgggact

SEQ ID NO: 90 - DNA, 40 bp, Homo sapiens
gggggtctcgcctcgggactacttgactggggtaaggtgg

SEQ ID NO: 91 - DNA, 20 bp, Homo sapiens
acaaaagaagatgccacagc

SEQ ID NO: 92 - DNA, 20 bp, Homo sapiens
tgcagcaacaaaaacacagt

SEQ ID NO: 93 - DNA, 41 bp, Homo sapiens
tgcagcaacaaaaacacagttcctagggagttgaataaggc

SEQ ID NO: 94 - DNA, 20 bp, Homo sapiens
tgataccccaactccctcta

SEQ ID NO: 95 - DNA, 20 bp, Homo sapiens
aaagcaggagggaacagagc

SEQ ID NO: 96 - DNA, 37 bp, Homo sapiens
aaagcaggagggaacagagcactgcagggaccacagg

SEQ ID NO: 97 - DNA, 20 bp, Homo sapiens
tgcccagctactgctaccta

SEQ ID NO: 98 - DNA, 18 bp, Homo sapiens
cccagttcaggtccagga

SEQ ID NO: 99 - DNA, 36 bp, Homo sapiens
cccagttcaggtccaggatgtcataccgagtcttct

SEQ ID NO: 100 - DNA, 20 bp, Homo sapiens
gccctgggactgatagcaag

SEQ ID NO: 101 - DNA, 20 bp, Homo sapiens
agacgaagcagaggggcaaa

SEQ ID NO: 102 - DNA, 40 bp, Homo sapiens
agacgaagcagaggggcaaaggggagttccaaaacacctg

SEQ ID NO: 103 - DNA, 24 bp, Homo sapiens
ccagaaatgggtcagaatggacaa

SEQ ID NO: 104 - DNA, 24 bp, Homo sapiens
catctgccggggtaggagaaagcc

SEQ ID NO: 105 - DNA, 44 bp, Homo sapiens
catctgccggggtaggagaaagcctgtctgctgcagagcctggc

SEQ ID NO: 106 - DNA, 18 bp, Homo sapiens
ctggagccccgaggaagc

SEQ ID NO: 107 - DNA, 18 bp, Homo sapiens
cactgggggtttcctttg

SEQ ID NO: 108 - DNA, 36 bp, Homo sapiens
cactgggggtttccttggaaggccagatcttctctt

SEQ ID NO: 109 - DNA, 25 bp, Homo sapiens
agtgcatctccatgtcccgctacta

SEQ ID NO: 110 - DNA, 25 bp, Homo sapiens
cgatgttcttaacgtggtgcatcaa

SEQ ID NO: 111 - DNA, 45 bp, Homo sapiens
cgatgttcttaacgtggtgcatcaacaggctgtggcttgctttgt

SEQ ID NO: 112 - DNA, 21 bp, Homo sapiens
cctcctgcagtcccagctctc

SEQ ID NO: 113 - DNA, 21 bp, Homo sapiens
ggtttctccccgccgttctca

SEQ ID NO: 114 - DNA, 42 bp, Homo sapiens
ggtttctccccgccgttctcatgagcaaataatccattctga

SEQ ID NO: 115 - DNA, 20 bp, Homo sapiens
ggagctcccctgtggtcatc

SEQ ID NO: 116 - DNA, 20 bp, Homo sapiens
ttttgaactgtggaaggaac

SEQ ID NO: 117 - DNA, 40 bp, Homo sapiens
ttttgaactgtggaaggaacaggggctcgctcttctgatt

SEQ ID NO: 118 - DNA, 25 bp, Homo sapiens
aaagtacttggagtctgcaggtgcg

SEQ ID NO: 119 - DNA, 25 bp, Homo sapiens
tgcaattgacctccagtgaagttca

SEQ ID NO: 120 - DNA, 50 bp, Homo sapiens
tgcaattgacctccagtgaagttcagtgccccacacaggaaaatagtctc

SEQ ID NO: 121 - DNA, 18 bp, Homo sapiens
cactgggacgaaggggaa

SEQ ID NO: 122 - DNA, 18 bp, Homo sapiens
gtcataagccccgccaat

SEQ ID NO: 123 - DNA, 36 bp, Homo sapiens
gtcataagccccgccaatagacacagctgccatcct

SEQ ID NO: 124 - DNA, 18 bp, Homo sapiens
aagcccaccgacccatct

SEQ ID NO: 125 - DNA, 18 bp, Homo sapiens
tcaggcgcctcacaaagc

SEQ ID NO: 126 - DNA, 36 bp, Homo sapiens
tcaggcgcctcacaaagcagtgctggggtaggtgaa

SEQ ID NO: 127 - DNA, 21 bp, Homo sapiens
ggtcggagtcaacggatttgg

SEQ ID NO: 128 - DNA, 21 bp, Homo sapiens
cctccgacgcctgcttcacca

SEQ ID NO: 129 - DNA, 42 bp, Homo sapiens
cctccgacgcctgcttcaccagaggggccatccacagtcttc

SEQ ID NO: 130 - DNA, 21 bp, Homo sapiens
gaggagcgaggactggagcca

SEQ ID NO: 131 - DNA, 21 bp, Homo sapiens
gcacctccatgggtcgaaatt

SEQ ID NO: 132 - DNA, 42 bp, Homo sapiens
gcacctccatgggtcgaaatttgtcagtgggtctctaataaa

SEQ ID NO: 133 - DNA, 20 bp, Homo sapiens
tcgccgaggagagcaagttc

SEQ ID NO: 134 - DNA, 20 bp, Homo sapiens
ggaacagcgcggtcctgtaa

SEQ ID NO: 135 - DNA, 40 bp, Homo sapiens
ggaacagcgcggtcctgtaagaataatccaaaagaccaga

SEQ ID NO: 136 - DNA, 20 bp, Homo sapiens
cgtaccctgtgccattccaa

SEQ ID NO: 137 - DNA, 20 bp, Homo sapiens
taaacagccagacagatgca

SEQ ID NO: 138 - DNA, 40 bp, Homo sapiens
taaacagccagacagatgcaatacggggaataaaccacgt

SEQ ID NO: 139 - DNA, 18 bp, Homo sapiens
gtcggtggcttctgctga

SEQ ID NO: 140 - DNA, 19 bp, Homo sapiens
aacccttgagtgtagccca

SEQ ID NO: 141 - DNA, 37 bp, Homo sapiens
aacccttgagtgtagcccagatgtgcatattcacctc

SEQ ID NO: 142 - DNA, 20 bp, Homo sapiens
gctgctggccgaaaacttgc

SEQ ID NO: 143 - DNA, 20 bp, Homo sapiens
gtctgccttcgttgctccca

SEQ ID NO: 144 - DNA, 40 bp, Homo sapiens
gtctgccttcgttgctcccatttcttccttgttagcacag

SEQ ID NO: 145 - DNA, 21 bp, Homo sapiens
gcagagccggggacaagagaa

SEQ ID NO: 146 - DNA, 21 bp, Homo sapiens
ctgctctttctctccattgac

SEQ ID NO: 147 - DNA, 42 bp, Homo sapiens
ctgctctttctctccattgacgctcttcctgtagtgcattca

SEQ ID NO: 148 - DNA, 20 bp, Homo sapiens
gggacgctcctgattatgac

SEQ ID NO: 149 - DNA, 20 bp, Homo sapiens
gcaaaccatggccgcttccc

SEQ ID NO: 150 - DNA, 40 bp, Homo sapiens
gcaaaccatggccgcttcccttctccaaaatgtccacacg

SEQ ID NO: 151 - DNA, 20 bp, Homo sapiens
gtgcgagtcgtctatggttc

SEQ ID NO: 152 - DNA, 20 bp, Homo sapiens
agttgtgtgcggaaatccat

SEQ ID NO: 153 - DNA, 40 bp, Homo sapiens
agttgtgtgcggaaatccattgctctgggtgatcttgttc

SEQ ID NO: 154 - DNA, 20 bp, Homo sapiens
tccgctgcaaatacatctcc

SEQ ID NO: 155 - DNA, 20 bp, Homo sapiens
tgtttcccgttgccattgat

SEQ ID NO: 156 - DNA, 40 bp, Homo sapiens
tgtttcccgttgccattgattaggacctcatggatcagca

SEQ ID NO: 157 - DNA, 20 bp, Homo sapiens
gctctacctggacctgctgt

SEQ ID NO: 158 - DNA, 20 bp, Homo sapiens
ggaacacagggaacatcacc

SEQ ID NO: 159 - DNA, 40 bp, Homo sapiens
ggaacacagggaacatcacctagagcaggatggccacact

SEQ ID NO: 160 - DNA, 20 bp, Homo sapiens
atgtgaaccagccagatgtt

SEQ ID NO: 161 - DNA, 20 bp, Homo sapiens
ctctgggttctctgccgtag

SEQ ID NO: 162 - DNA, 40 bp, Homo sapiens
ctctgggttctctgccgtagaggaggagggtggggctgag

SEQ ID NO: 163 - DNA, 23 bp, Homo sapiens
gctctacgttgcccgccagcctg

SEQ ID NO: 164 - DNA, 23 bp, Homo sapiens
gtttggggccgtctttgtagtaa

SEQ ID NO: 165 - DNA, 46 bp, Homo sapiens
gtttggggccgtctttgtagtaatttattatgctgttgacggtttg

SEQ ID NO: 166 - DNA, 20 bp, Homo sapiens
ttttgggagggggttgtgcc

SEQ ID NO: 167 - DNA, 20 bp, Homo sapiens
ggccacaccagcagcatcca

SEQ ID NO: 168 - DNA, 40 bp, Homo sapiens
ggccacaccagcagcatccataaccaacttctgaggaact

SEQ ID NO: 169 - DNA, 20 bp, Homo sapiens
agttgtggcctttacagcag

SEQ ID NO: 170 - DNA, 20 bp, Homo sapiens
tgtgtgccccaagtaatttt

SEQ ID NO: 171 - DNA, 40 bp, Homo sapiens
tgtgtgccccaagtaattttgcacggacaattttaaaggg

SEQ ID NO: 172 - DNA, 21 bp, Homo sapiens
cctaccagctccagacctttg

SEQ ID NO: 173 - DNA, 21 bp, Homo sapiens
tggcttcgtcagaatcacgtt

SEQ ID NO: 174 - DNA, 42 bp, Homo sapiens
tggcttcgtcagaatcacgttcccagtattactgcacacgtc

SEQ ID NO: 175 - DNA, 22 bp, Homo sapiens
ccttactgtgagtctgggttga

SEQ ID NO: 176 - DNA, 22 bp, Homo sapiens
tgggttttctgctttctgatat

SEQ ID NO: 177 - DNA, 44 bp, Homo sapiens
tgggttttctgctttctgatatgtccctttacagcagtcatgtg

SEQ ID NO: 178 - DNA, 19 bp, Homo sapiens
tggcccagctcaaacagaa

SEQ ID NO: 179 - DNA, 19 bp, Homo sapiens
cctcttcccctccctgtta

SEQ ID NO: 180 - DNA, 37 bp, Homo sapiens
cctcttcccctccctgttacttgtaaacgtcgaggtg

SEQ ID NO: 181 - DNA, 20 bp, Homo sapiens
ctcaaggatgccaggaacaa

SEQ ID NO: 182 - DNA, 20 bp, Homo sapiens
acactgagcccaccacctag

SEQ ID NO: 183 - DNA, 40 bp, Homo sapiens
acactgagcccaccacctagccactgccatatccagagga

SEQ ID NO: 184 - DNA, 20 bp, Homo sapiens
tgccttggacacggggttct

SEQ ID NO: 185 - DNA, 20 bp, Homo sapiens
ttgcccttctgaatagtccc

SEQ ID NO: 186 - DNA, 40 bp, Homo sapiens
ttgcccttctgaatagtccccatggatgccgtctaattgc

SEQ ID NO: 187 - DNA, 20 bp, Homo sapiens
ttcagcgagagcagcgacac

SEQ ID NO: 188 - DNA, 20 bp, Homo sapiens
cagaaccaacagggagaacc

SEQ ID NO: 189 - DNA, 40 bp, Homo sapiens
cagaaccaacagggagaacctcttgatgctggtgctggaa

SEQ ID NO: 190 - DNA, 20 bp, Homo sapiens
tgacatcgaggtggagagcg

SEQ ID NO: 191 - DNA, 20 bp, Homo sapiens
cccccatcgaaggcagaaat

SEQ ID NO: 192 - DNA, 40 bp, Homo sapiens
tgacatcgaggtggagagcgcacacacaccagcaagatat

SEQ ID NO: 193 - DNA, 21 bp, Homo sapiens
cctgctgaagtggctgccaaa

SEQ ID NO: 194 - DNA, 21 bp, Homo sapiens
aacttggtttgatgctgtgcc

SEQ ID NO: 195 - DNA, 42 bp, Homo sapiens
aacttggtttgatgctgtgcctttcttcctggagactcaaaa

SEQ ID NO: 196 - DNA, 25 bp, Homo sapiens
tgtccgcagttgatggccagagaca

SEQ ID NO: 197 - DNA, 25 bp, Homo sapiens
acttgattaccgcagacagtgatga

SEQ ID NO: 198 - DNA, 50 bp, Homo sapiens
acttgattaccgcagacagtgatgaacaaccggttgaggtcctgataaat

SEQ ID NO: 199 - DNA, 21 bp, Homo sapiens
tgctgaagaacggagggatgt

SEQ ID NO: 200 - DNA, 21 bp, Homo sapiens
tttgccattttcctgctcctc

SEQ ID NO: 201 - DNA, 42 bp, Homo sapiens
tttgccattttcctgctcctccatctccaaaaaaagtcttcg

SEQ ID NO: 202 - DNA, 20 bp, Homo sapiens
caggagttcgcagtcaagat

SEQ ID NO: 203 - DNA, 20 bp, Homo sapiens
acaggatgttctccggcttg

SEQ ID NO: 204 - DNA, 40 bp, Homo sapiens
acaggatgttctccggcttgatctggcttgcttccgactc

SEQ ID NO: 205 - DNA, 20 bp, Homo sapiens
acaattgactctggccttcc

SEQ ID NO: 206 - DNA, 20 bp, Homo sapiens
tagacaatggccagcgcaac

SEQ ID NO: 207 - DNA, 40 bp, Homo sapiens
acaattgactctggccttccacgatctcagacgtcagcgt

SEQ ID NO: 208 - DNA, 20 bp, Homo sapiens
gagagcccggacatcaagta

SEQ ID NO: 209 - DNA, 20 bp, Homo sapiens
acttccctttgccctggtag

SEQ ID NO: 210 - DNA, 40 bp, Homo sapiens
acttccctttgccctggtagactgagacatcttccctcca

SEQ ID NO: 211 - DNA, 21 bp, Homo sapiens
cgctcatcgtgggtctcctaa

SEQ ID NO: 212 - DNA, 21 bp, Homo sapiens
agaagtcctgggcattgtcgg

SEQ ID NO: 213 - DNA, 42 bp, Homo sapiens
agaagtcctgggcattgtcggcaggtcggccaggtcatactc

SEQ ID NO: 214 - DNA, 20 bp, Homo sapiens
cctgctccgctgctccttgg

SEQ ID NO: 215 - DNA, 20 bp, Homo sapiens
catgcccaacactcccctcc

SEQ ID NO: 216 - DNA, 40 bp, Homo sapiens
catgcccaacactcccctccccctctctaacacctcagca

SEQ ID NO: 217 - DNA, 20 bp, Homo sapiens
aggtacagctccccaccagc

SEQ ID NO: 218 - DNA, 20 bp, Homo sapiens
cttccagccagggcctgagc

SEQ ID NO: 219 - DNA, 40 bp, Homo sapiens
cttccagccagggcctgagcaggaatggttaccgtttgcc

SEQ ID NO: 220 - DNA, 20 bp, Homo sapiens
ggagcccaactgcgccgacc

SEQ ID NO: 221 - DNA, 22 bp, Homo sapiens
ccttcggtgactgatgatctaa

SEQ ID NO: 222 - DNA, 38 bp, Homo sapiens
ggagcccaactgcgccgacccccgtggacctggctgag

SEQ ID NO: 223 - DNA, 21 bp, Homo sapiens
gcgctgcaggttatgaaactt

SEQ ID NO: 224 - DNA, 21 bp, Homo sapiens
agccccgtttgcctgcatcag

SEQ ID NO: 225 - DNA, 42 bp, Homo sapiens
agccccgtttgcctgcatcagacccggaggtggccttctttg

SEQ ID NO: 226 - DNA, 20 bp, Homo sapiens
gcatcccgacgccctcaacc

SEQ ID NO: 227 - DNA, 20 bp, Homo sapiens
gatgtccacgaggtcctgag

SEQ ID NO: 228 - DNA, 40 bp, Homo sapiens
gcatcccgacgccctcaaccgatgtccacgaggtcctgag

SEQ ID NO: 229 - DNA, 20 bp, Homo sapiens
gctgtccctcccccttgtct

SEQ ID NO: 230 - DNA, 20 bp, Homo sapiens
tgttccgctgctaatcaaag

SEQ ID NO: 231 - DNA, 40 bp, Homo sapiens
tgttccgctgctaatcaaagtactcccccatcatataccc

SEQ ID NO: 232 - DNA, 20 bp, Homo sapiens
cgggacttggagaagcactg

SEQ ID NO: 233 - DNA, 20 bp, Homo sapiens
tagaagaatcgtcggttgca

SEQ ID NO: 234 - DNA, 40 bp, Homo sapiens
tagaagaatcgtcggttgcatgacatcctggctctcctgc

SEQ ID NO: 235 - DNA, 20 bp, Homo sapiens
agaccggcgcacagaggaag

SEQ ID NO: 236 - DNA, 20 bp, Homo sapiens
ctttttggacttcaggtggc

SEQ ID NO: 237 - DNA, 40 bp, Homo sapiens
ctttttggacttcaggtggccctcattcagctctcggaac

SEQ ID NO: 238 - DNA, 23 bp, Homo sapiens
cgaaggctacgaaggctattaca

SEQ ID NO: 239 - DNA, 23 bp, Homo sapiens
tggggagaagaaggggaccacga

SEQ ID NO: 240 - DNA, 46 bp, Homo sapiens
tggggagaagaaggggaccacgaaggaatcctgggagatacaagaa

SEQ ID NO: 241 - DNA, 22 bp, Homo sapiens
gctccagcggtgtaaacctgca

SEQ ID NO: 242 - DNA, 22 bp, Homo sapiens
cgtgcaaattcaccagaaggca

SEQ ID NO: 243 - DNA, 44 bp, Homo sapiens
cgtgcaaattcaccagaaggcatcaacttcatttcatagtctga

SEQ ID NO: 244 - DNA, 20 bp, Homo sapiens
ctttcggttttcagggggaa

SEQ ID NO: 245 - DNA, 20 bp, Homo sapiens
tggctcacagttctgcaggc

SEQ ID NO: 246 - DNA, 43 bp, Homo sapiens
tggctcacagttctgcagcaggcaattccttatggcgcacagg

SEQ ID NO: 247 - DNA, 20 bp, Homo sapiens
ggggtcccgctcatcaagta

SEQ ID NO: 248 - DNA, 20 bp, Homo sapiens
aactgccacatcctttgcgt

SEQ ID NO: 249 - DNA, 40 bp, Homo sapiens
aactgccacatcctttgcgtccgcatgaagatgggagctc

SEQ ID NO: 250 - DNA, 21 bp, Homo sapiens
gcttccaagcccgacctgatg

SEQ ID NO: 251 - DNA, 21 bp, Homo sapiens
acggtggaaatggtagtagga

SEQ ID NO: 252 - DNA, 40 bp, Homo sapiens
acggtggaaatggtagtaggactccagccctgagggttcc

SEQ ID NO: 253 - DNA, 20 bp, Homo sapiens
ggccagaatttagcaagaca

SEQ ID NO: 254 - DNA, 20 bp, Homo sapiens
tgactatgggcctagagcag

SEQ ID NO: 255 - DNA, 41 bp, Homo sapiens
tgactatgggcctagagcagggcttcttcttttccactggt

SEQ ID NO: 256 - DNA, 18 bp, Homo sapiens
ccgaccagatcaccctcc

SEQ ID NO: 257 - DNA, 18 bp, Homo sapiens
gcttccgcacgtagacct

SEQ ID NO: 258 - DNA, 36 bp, Homo sapiens
gcttccgcacgtagacctagccccgtctccgcatca

SEQ ID NO: 259 - DNA, 24 bp, Homo sapiens
tttcagaaggtctgccaacaccaa

SEQ ID NO: 260 - DNA, 24 bp, Homo sapiens
gtgtccaccaaggtcctgagatcc

SEQ ID NO: 261 - DNA, 48 bp, Homo sapiens
gtgtccaccaaggtcctgagatcccatttctgccagtttctgctgaaa

SEQ ID NO: 262 - DNA, 20 bp, Homo sapiens
aggtgggcaaagggaagtaa

SEQ ID NO: 263 - DNA, 20 bp, Homo sapiens
tagagcccctgagaagagcc

SEQ ID NO: 264 - DNA, 40 bp, Homo sapiens
tagagcccctgagaagagcccagagaactgacagtccgtg

SEQ ID NO: 265 - DNA, 21 bp, Homo sapiens
ccagccactgttgcagcatga

SEQ ID NO: 266 - DNA, 21 bp, Homo sapiens
aggcaaatgggactcatacac

SEQ ID NO: 267 - DNA, 42 bp, Homo sapiens
aggcaaatgggactcatacacgggctggtgctggagtgacta

SEQ ID NO: 268 - DNA, 20 bp, Homo sapiens
gctctgagcgagattgagac

SEQ ID NO: 269 - DNA, 20 bp, Homo sapiens
caggatcacacagcagatga

SEQ ID NO: 270 - DNA, 40 bp, Homo sapiens
caggatcacacagcagatgatccacatagtctaccgcgtg

SEQ ID NO: 271 - DNA, 20 bp, Homo sapiens
tgtgcacaaatccatcaacc

SEQ ID NO: 272 - DNA, 20 bp, Homo sapiens
gaaaggctccagggttaggt

SEQ ID NO: 273 - DNA, 40 bp, Homo sapiens
gaaaggctccagggttaggtcacggatccgcatggccatc

SEQ ID NO: 274 - DNA, 20 bp, Homo sapiens
ccacgctcttctgcctgctg

SEQ ID NO: 275 - DNA, 20 bp, Homo sapiens
ctggtaggagacggcgatgc

SEQ ID NO: 276 - DNA, 40 bp, Homo sapiens
ctggtaggagacggcgatgcgggtttgctacaacatgggc

SEQ ID NO: 277 - DNA, 20 bp, Homo sapiens
gccgcctacttggtgctaac

SEQ ID NO: 278 - DNA, 20 bp, Homo sapiens
cgtgctgcgccctgccttat

SEQ ID NO: 279 - DNA, 40 bp, Homo sapiens
cgtgctcgcccctgccttagaggagtgccaagtttctatt

SEQ ID NO: 280 - DNA, 21 bp, Homo sapiens
gattcctatgtgggcgacgag

SEQ ID NO: 281 - DNA, 20 bp, Homo sapiens
ccatctcttgctcgaagtcc

SEQ ID NO: 282 - DNA, 40 bp, Homo sapiens
ccatctcttgctcgaagtccgccagccaggtccagacgca

(19) United States
(12) Patent Application Publication
(10) Pub. No.: US 2003/0186246 A1
(43) Pub. Date: Oct. 2, 2003

(54) Multiplex standardized reverse transcriptase-polymerase chain reacton method for assessment of gene expression in small biological samples

(76) Inventors: James C. Willey, Toledo, OH (US); Erin L. Crawford, Rossford, OH (US)

Correspondence Address: EMCH, SCHAFFER, SCHAUB & PORCELLO CO, P O BOX 916, ONE SEAGATE SUITE 1980, TOLEDO, OH, 43697

(21) Appl. No.: 10/109,349

(22) Filed: Mar. 28, 2002

(51) Int. Cl. C12Q 1/68; C12P 19/34

(52) U.S. Cl. 435/6; 435/91.2

(57) ABSTRACT

A method for direct comparison of numerical gene expression values between samples of genes using reverse transcription-polymerase chain reaction is described. cDNA, a competitive template mixture, and primer pairs for a plurality of genes are combined with at least one suitable buffer and at least one suitable enzyme to form a mixture. The mixture is amplified for a predetermined number of cycles to form PCR products. The PCR products are mixed with at least one suitable buffer, at least one enzyme, and one primer pair specific for each of the genes. The resulting mixture is amplified an additional predetermined number of cycles.

This invention was made under Research Grant No. NIH CA85147 who may have certain rights thereto.

### TECHNICAL FIELD

The present invention relates to a multiplex standardized reverse transcriptase polymerase chain reaction method for assessment of gene expression in small biological samples. The method is useful to assess small biological samples, such as fine needle aspirate biopsies, and laser captured microdissected materials. Without the method described here, such samples could be assessed for only a small number of genes. Use of the method described herein allows for the standardized measurement of hundreds of genes from the same sample that, in the past, could be assessed for only one gene.

### BACKGROUND OF THE INVENTION

The PCR techniques are generally described in U.S. Pat. Nos. 4,683,195; 4,683,202; and 4,965,188. The PCR technique generally involves a process for amplifying any desired specific nucleic acid sequence contained within a nucleic acid molecule. The PCR process includes treating separate complementary strains of the nucleic acid with an excess of two oligonucleotide primers. The primers are extended to form complementary primer extension products which act as templates for synthesizing the desired nucleic acid sequence. The PCR process is carried out in a simultaneous step-wise fashion and can be repeated as often as desired in order to achieve increased levels of amplification of the desired nucleic acid sequence. According to the PCR process, the sequences of DNA between the primers on the respective DNA strains are amplified selectively over the remaining portions of the DNA and selected sample. The PCR process provides for the specific amplification of a desired region of DNA.

The yield of product from PCR increases exponentially for an indefinite number of cycles. At some point and for uncertain reasons, the reaction becomes limited and PCR product increases at an unknown rate. Consequently, the yield of amplified product has been reported to vary by as much as 6-fold between identical samples run simultaneously. (Gilliland, G., et al., Proc. Natl. Acad. Sci. 87:2725-2729, 1990). (These publications and other reference materials have been included to provide additional details on the background of the invention and, in particular instances, the practice of the invention, and all are expressly incorporated herein by reference). Therefore, after a certain number of PCR cycles, the initial concentrations of target DNA cannot be accurately determined by extrapolation. In an attempt to make PCR quantitative, various investigators have analyzed samples amplified for a number of cycles known to provide exponential amplification (Horikoshi, T., et al., Cancer Res. 52:108-116 (1992); Noonan, K. E., et al., Proc. Natl. Acad. Sci. 87:7160-7164 (1990); Murphy, L. D., et al., Biochemistry 29:10351-10356 (1990); Carre, P. C., et al., J. Clin. Invest. 88:1802-1810 (1991); Chelly, J., et al., Eur. J. Biochem 187:691-698 (1990); Abbs, S., et al., J. Med. Genet. 29:191-196 (1992); Feldman, A. M. et al., Circulation 83:1866-1872 (1991). In general, these analyses are done early in the PCR process prior to the endpoint, when the PCR product yield is small. Consequently, more starting cDNA must be included in the PCR reaction for the product to reach quantifiable levels. Also, the exponential phase must be defined for each set of experimental conditions, requiring additional cost in time and materials.

Another development is competitive PCR, wherein PCR is conducted in the presence of single base mutated competitive templates (Gilliland, supra; Becker-Andre, et al., Nucleic Acids Res. 17:9437-9446 (1989)). A known amount of competitive template is co-amplified with an unknown amount of target sequence. The competitor is the same sequence (except for single base mutation or deletion of a portion of the sequence) as the target, uses the same primers for amplification as the target cDNA, and amplifies with the same efficiency as the target cDNA. The starting ratio of target/standard is preserved throughout the entire amplification process, even after the exponential phase is complete.

Competitive PCR is discussed in general in Siebert, P. D., et al., Nature 359:557-558 (1992); Siebert, P. D., et al., BioTechniques 14:244-249 (1993), and Clontech Brochure, 1993, Reverse Transcriptase-PCR (RT-PCR). However, competitive PCR alone does not adequately control for variation in starting amounts of template. Degradation of samples and pipetting errors can lead to variation.

When using Northern analysis to measure gene expression, it is possible to overcome these problems by probing the same blot for both a target gene and a “housekeeping” or reference gene which is not expected to vary among tissue samples or in response to stimuli. The reference gene acts as a denominator in determining the relative expression of a target gene. In attempts to apply this concept, other investigators have PCR-amplified in separate tubes. However, when the two genes are amplified in separate tubes, intertube variation in amplification conditions and pipetting errors are unavoidable. While non-competitive multiplex PCR, where the target and reference gene are amplified in the same tube, has also been described in Noonan, supra, this method is inconvenient because it requires the generation of standard curves to determine the exponential range of amplification nuclides.

An alternative approach, real-time RT-PCR, determines the log-linear phase of amplification automatically. However, real-time RT-PCR still requires standard curves in order to compare expression of one gene to another.

The Willey and Willey et al. U.S. Pat. Nos. 5,043,390; 5,639,606; and 5,876,978, which are expressly incorporated herein by reference, describe quantitative measurement of gene expression techniques which have none of the above-described drawbacks and which can be performed by a technician with standard training.

The present invention is an improvement upon the above Willey and Willey et al. '390, '606 and '978 PCR amplification processes that allows simultaneous amplification of a “target gene”, a “housekeeping” or reference gene and competitive templates for each of these genes. The terms “target DNA sequence” and “target gene” generally refer to a gene of interest for which there is a desire to selectively amplify that gene or DNA sequence. The terms “housekeeping” or “reference” gene refers to genes that are suitable references, for amount of RNA per PCR reaction.

In a general and overall sense, a key is the simultaneous use of primers for a target gene, primers for a housekeeping or reference gene, and two internal standard competitive templates comprising mutants of the target gene and reference gene. These mutations can be point mutations, insertions, deletions or the like.

The Willey and Willey et al. '390, '606 and '978 patents are directed to a method for quantifying the amount of a target DNA sequence within an identified region of a selected cDNA molecule that is present within a heterogeneous mixture of cDNA molecules. More than one targeted gene and/or reference gene can be utilized. The quantitation of such additional target and/or housekeeping genes necessitates the further inclusion of an internal standard competitive template comprising a mutation of that additional target and/or housekeeping gene. It is to be understood that the mutated competitive templates comprise at least one nucleotide that is mutated relative to the corresponding nucleotide of the target sequence. Mutation of at least one single nucleotide that is complementary to the corresponding nucleotide of the housekeeping gene sequence is required. However, it is understood that longer deletions, insertions or alterations are also useful. The target gene primers (which serve as primers for both the native and competitive templates of the target gene), housekeeping gene primers (which serve as primers for both the native and competitive template of the housekeeping gene), competitive template of the target gene, and competitive template of the housekeeping gene are subjected to a PCR process along with native cDNA which contains the DNA for both the target gene and the housekeeping gene.

The PCR process provides cDNA products of 1) native cDNA of the target gene and the housekeeping gene and 2) mutated competitive template cDNA of the target gene and the housekeeping gene. The cDNA products are isolated using methods suitable for isolating cDNA products. The relative presence of the native cDNA products and the mutated cDNA products are detected by measuring the amounts of native cDNA coding for the target gene and mutated cDNA coding for the competitive template of the target gene as compared to the amounts of native cDNA coding for the housekeeping gene and mutated cDNA coding for competitive template of the housekeeping gene.

According to the present invention herein “a sample” generally indicates a sample of tissue or fluid isolated from a plant, individual or animal in vitro cell culture constituents.

The terms “primers”, “nucleic acids” and “oligonucleotides” are understood to refer to polyribonucleotides and polydeoxyribonucleotides and there is no intended distinction in the length of sequences referred to by these terms. Rather, these terms refer to the primary structure of the molecule. These terms include double and single stranded RNA and double and single stranded DNA. It is to be understood that the oligonucleotides can be derived from any existing or natural sequence and generated in any manner. It is further understood that the oligonucleotides can be generated from chemical synthesis, reverse transcription, DNA replication and a combination of these generating methods.

The term “primer” generally refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. It is understood that while a primer is preferably a single strand sequence, such that amplification efficiency is optimized, other double stranded sequences can be practiced with the present invention.

The terms “target gene”, “sequence” or “target nucleic acid sequence” are meant to refer to a region of an oligonucleotide, which is either to be amplified and/or detected. It is to be understood that the target sequence resides between the primer sequences used in the amplification process.

The Willey and Willey et al. '490, '606 and '978 patents also describe the PCR amplification of a) cDNA from at least one target gene of interest and at least one “housekeeping” gene and b) competitive templates comprising sequences of the target gene of interest and the “housekeeping” gene that have been artificially shortened. These shortened sequences retain sequences homologous to both the target gene and the housekeeping gene primers used in PCR amplification. RNA extracted from sample cells or tissues are reverse transcribed. Serial dilutions of cDNA are PCR amplified in the presence of oligonucleotides homologous to the target gene and the “housekeeping” gene, and quantified amounts of internal mutated standard competitive templates. The amplified DNA may be restriction digested and electrophoresed on an agarose gel stained with ethidium bromide, or other electrophoresis method such as Agilent or AB1 310, separating native from mutated products. Densitometry is performed to quantify the bands. This technique to measure the relative expression of a target gene to a “housekeeping” gene is precise and reproducible for studies done with the same master mixture and dilution of internal standards. When replicate assessments of gene expression on a particular sample are conducted, the standard deviation is generally less than about 50% of the mean. This technique is useful to measure changes in gene expression. This method is particularly useful when the amount of study sample is limited or the level of gene expression is low.

These improvements are important because recent progress in the Human Genome Project has added greatly to our knowledge and increased the opportunity for correlating genetic basis for known phenotypes. Measurement of gene expression patterns which improves understanding of normal development as well as many disease processes, is achieved readily by using the standardized RT-PCR (StaRT PCR) reverse transcriptase-polymerase chain reaction which is described in detail in the Willey and Willey et al. U.S. Pat. Nos. 5,876,978, 5,639,606 and 5,643,765.

One primary advantage of the StaRT-PCR process is the ability to rapidly and reproducibly attain standardized, quantitative data for many genes simultaneously. Each gene expression measurement is reported in a numerical value that allows for the combination of values into indices and for direct inter-experiment comparison. Since the data are standardized against a common internal control, it is also possible to make direct comparisons between samples and between laboratories.

However, in order to correlate gene expression patterns with clinically relevant phenotypes, it may be necessary to evaluate expression levels of about 50-100 genes. In addition, these gene expression patterns may need to be evaluated in small and precious samples.

The size of biopsies obtained in many clinical situations has been decreasing over the years as cytologic methods have improved and economic pressures to reduce costs have increased. For example, biopsies of suspected cancerous lesions in the lung, breast, prostate, thyroid, and pancreas, commonly are done by fine needle aspirate (FNA) biopsy. In addition, there is a need to evaluate expression patterns in samples from anatomically small, but functionally important tissues of the brain, developing embryo, and animal models, including laser captured micro-dissected samples and flow-sorted cell populations. In addition, because it may be necessary to measure 50-100 genes to fully characterize a phenotype (Heldenfalk, I. et al. NEJM 344: 539, 2000), it is important to reduce consumption of cDNA and the cost of each assay as much as possible.

Recent advances in automation and miniaturation have made it possible to greatly reduce PCR reaction volumes and therefore decrease consumption of reagents and samples. It is important though, to ensure enough cDNA is used in each reaction to detect rare transcripts and that the relationship of one transcript to another is not altered by the detection method.

Therefore, there is a need for an improved variation of the StaRT-PCR process. There is also a need to use significantly less cDNA per gene expression assay and yet maintain sensitivity to detect rare transcripts.

In particular, these needs are shown in recent studies in which StaRT-PCR was used to identify patterns of gene expression associated with lung cancer (Crawford, E. L. et al. Normal bronchial epithelial cell expression of glutathione transferase P1, glutathione transferase M3, and glutathione peroxidase is low in subjects with bronchogenic carcinoma. Cancer Res., 60: 1609-1618, 2000; DeMuth, et al., The gene expression index c-myc x E2F-1/p21 is highly predictive of malignant phenotype in human bronchial epithelial cells. Am.J.Respir.Cell Mol.Biol., 19: 18-24, 1998); pulmonary sarcoidosis (Allen, J. T., et al., Enhanced insulin-like growth factor binding protein-related protein 2 (connective tissue growth factor) expression in patients with idiopathic pulmonary fibrosis and pulmonary sarcoidosis. Am. J. Respir. Cell Mol. Biol., 21: 693-700, 1999); cystic fibrosis (Allen, et al, supra); and chemoresistance in childhood leukemias (Rots, M. G., et al., Circumvention of methotrexate resistance in childhood leukemia subtypes by rationally designed antifolates. Blood, 94(9): 3121-3128,1999; Rots, M. G., et al., mRNA expression levels of methotrexate resistance-related proteins in childhood leukemia as determined by a competitive template-based RT-PCR method. Leukemia, 14:2166-2175 (2000).

As the throughput capacity for gene expression measurement increases with implementation of robots and capillary electrophoreses (CE) devices, it is important to develop methods that will allow a reduction in the amount of cDNA and other reagents required.

One way to accomplish this would be to multiplex amplify many genes in each PCR reaction. It is possible to StaRT-PCR amplify native templates (NTs) and competitive templates (CTs) for two genes in a single PCR reaction, but, until the present invention, efforts with more than two genes in a single reaction have not resulted in quantifiable bands.

The present invention provides an improvement of the StaRT-PCR process in which the cDNA is PCR amplified in two rounds. In the first round, primers for multiple genes are present along with cDNA and a CT mix containing CTs for the same genes. In round two, an aliquot of the round one amplification products are further amplified with primers for only one gene.

It is, therefore, an object of the present invention to provide an improved method for quantitative measurement of gene expression.

It is a further object of the present invention to provide a method for quantitative PCR-based measurement of gene expression that is suitable as a commercial process.

It is a further object of the present invention to provide a method to accurately and efficiently correlate gene expression patterns with clinically relevant phenotypes.

These and other objects, features and many of the attendant advantages of the invention will be better understood upon a reading of the following detailed description when considered in connection with the accompanying drawings herein.

### SUMMARY OF THE INVENTION

The present invention is directed to a multiplex standardized RT-PCR (StaRT-PCR) process that allows for the direct comparison of numerical gene expression values between samples and between laboratories. In one aspect, the present invention relates to a novel multiplex StaRT-PCR process that allows for the measurement of a substantially greater number of gene expression values without using increased amounts of cDNA and without compromising ability to detect rare transcripts in a statistically significant manner.

The multiplex StaRT-PCR method of the present invention is conducted using two rounds of amplification. In round one, cDNA, competitive template (CT) mixture and primer pairs for a desired number of genes (for example, 9 or 96 genes) are combined with buffer and enzyme and amplified for a desired number of cycles (for example, between about 3 to about 40 and in certain embodiments, for about 5, 8, 10 or 35 cycles) to form PCR products. In round two, the PCR products from round one are used, aliquots of the PCR products from round one are placed in new reaction tubes with buffer, enzyme and a primer pair specific for 1 of the desired number of genes used in round one and amplified for a predetermined number of additional cycles (for example, for an additional 35 cycles). No additional cDNA or CT mixture is added to this second reaction.

PCR products from round one can be diluted as much as 100,000-fold and still be quantified following amplification in round two. In contrast, a 100,000-fold dilution of the cDNA and the CT mixture used in round one followed by one round of 35 cycles with one primer pair did not yield any detectable product. Thus, using two rounds of amplification, the same amounts of cDNA and CT mixture that typically are used to obtain one gene expression measurement when only one round of amplification is used can be used to obtain 100,000 gene expression measurements without loss of sensitivity to detect rare transcripts. No significant differences between the gene expression values obtained by this method and the values obtained by control reactions were detected.

According to the present invention the numerical gene expression value is correlated with clinically relevant phenotypes which allows a combination of the gene expression values into at least one index that defines at least one specific phenotype. An internal standard competitive template is prepared for each gene and is cloned to generate competitive templates for at least about 10¹⁰ assays. In certain embodiments, the competitive templates for up to about 1000 genes are mixed together. Further, the assays can have a sensitivity of about 6 molecules or less.

In a preferred aspect, the CT mixture comprises a CT for at least one reference, or housekeeping gene and a CT for at least one target gene. Alternatively, the CT mixture comprises a CT for least one reference gene and a combination of CTs for multiple target genes.

The gene expression is quantified by calculating: i) a ratio of native template (NT) to competitive template (CT) for a reference gene; ii) ratios of NT/CT for each target gene; and iii) a ratio of the step (ii) ratio to the step (i) ratio.

In certain aspects, the method further includes use of high density oligonucleotide or cDNA arrays to measure PCR products following quantitative RT-PCR. The oligonucleotides or cDNA hybridizing to the sense strand or reverse strand of each cDNA being amplified is fluorescently labeled. Also, one or more of the dNTP's within the oligonucleotide in the PCR reaction can be labeled with a fluorescent dye. The expression of the target genes are quantified by comparing the fluorescent intensities of the spots in the array for the NT and CT for the reference gene and each target gene.

The high density oligonucleotide arrays have the following properties; for each gene, two loci on at least one oligonucleotide array are prepared; i) one locus on an array has attached to it oligonucleotides that are homologous to, and will bind to, sequences unique to the native template for the gene that was PCR-amplified; and, ii) another locus on the array has attached to it oligonucleotides that are homologous to, and will bind to, sequences that span the juncture between the 5′ end of the competitive template, and the truncated, mis-aligned 3′ end of the competitive template.

In another aspect, the invention relates to a system for quantitatively measuring gene expression of a plurality of target genes of interest of using the method described above and further performing the steps of: a) determining a desired concentration of CT reagents to be used when conducting a competitive form of reverse transcription-polymerase chain reaction (RT-PCR) of samples that target genes; b) selecting and causing to be dispensed at least one desired reagent into a plurality of reaction chambers in which the RT-PCR process is to be conducted; and is sent to a suitable device for identifying and/or labeling, for example by flowing the capillary electrofluoresis (CE) machine; and sometimes ending the process there. In certain embodiments, information from the CE machine is sent to step c) for analyzing quantitative data about the resultant product to quantitatively measure the expression of the target gene. The information from step c) can be provided in a “Report”, sent to a “Database”, and/or sent to step d) which reiterates the process for further analysis of the data.

In yet another aspect, the present invention relates to a computer program product for quantitatively measuring gene expression of target genes of interest through a two-step quantitative RT-PCR process. The computer program product includes a computer readable medium and instructions, stored on the computer readable medium, for quantitatively measuring gene expression. The instructions are used to carry out the steps described above.

The software program and product can also include instruction to dispense PCR reaction mixtures into high density cDNA and/or oligonucleotide arrays to measure PCR products following quantitative RT-PCR.

The computer program and product can include instructions to fluorescently label the oligonucleotide hybridizing the sense strand and/or anti-sense strand of each cDNA being amplified.

The computer program and product can include instructions to further label with a fluorescent dye one or more of the dNTP's within the oligonucleotide in the PCR reaction.

The computer program and product can include instruction where the target genes are quantified by comparing the fluorescent intensities of the arrays for the native and CT for the reference or housekeeping genes and targets genes.

The present invention also relates to a computer implemented method for quantitatively measuring gene expression of a plurality of target genes of interest using a two-step RT-PCR process. The method includes the steps of:

a) determining a desired concentration of CT reagents to be used when conducting a competitive form of reverse transcription-polymerase chain reaction (RT-PCR) of samples the target genes;

b) selecting and causing to be dispensed desired reagents into a plurality of reaction chambers in which to conduct the RT-PCR; and flows, or is sent to, a capillary electrofluoresis (CE) machine.

In certain embodiments, the information is sent on to a step c) for analyzing quantitative data about the resultant product to quantitatively measure the expression of the target genes; generating a “Report”, being stored in a “database;” and/or, further analysis.

In yet another aspect, the present invention includes optimizing the quantitative measurement of the genes using a further step:

d) of analyzing the data received in step (c) to determine whether the calculated ratio is within a desired range (for example, within a 10 fold ratio).

If the calculated ratio is not within the desired range, a new desired concentration of CT reagents (i.e., different from the original concentration selected in step (a)), is chosen and the steps (b)-(c) are repeated with the new concentration of CT reagents.

### DESCRIPTION OF THE FIGURES

FIG. 1 is a graph showing the minimum number of cells/PCR reaction needed to detect genes expressed at different levels with statistical confidence. The number of cells represented in a PCR reaction is shown along the X-axis. Initial copy number of mRNA transcripts loaded into a PCR reaction is shown on the Y-axis. It is assumed that at least 10 initial copies must be present in order to measure gene expression with statistical confidence and reduce the role of chance variation. A typical uniplex StaRT-PCR reaction contains cDNA from 100-1,000 cells. With this amount, one would expect cDNA from 100-1,000 cells to contain 10 transcripts for genes expressed at 0.1-1 transcript/cell. In human lung tissues, previously measured genes were expressed at 0.1 transcripts/cell (such as GADD45) to 10,000 transcripts/cell (such as CC10).

FIG. 2 is a table which shows the mean gene expression in cDNA derived from Stratagene Human Reference RNA (Seq. ID. Nos. 1-282) as measured by uniplex and multiplex StaRT-PCR for the genes listed therein. The “longer” name for p21 is “CDKN1A”. The sequences are listed in the same order as in Table 2. Each gene has 3 sequences. The first sequence is the forward primer, the second sequence is the reverse primer, and the third sequence is the CT primer. So, for example, sequence 1 HSD11B1 forward primer, sequence 2 is HSD11B1 reverse primer.

FIG. 3 is a table which shows the use of the multiplex StaRT-PCR process to increase amount of product without altering measurement of gene expression. Multiplex PCR reactions were amplified in the Rapidcycler. In round one, a 10 μl reaction mixture was prepared containing buffer, MgCl₂, dNTPs, a previously prepared mixture of cDNA and CT mixture (1:1 cDNA from A549 p85 and G.E.N.E. system 1 mix D), Taq polymerase and 1 μl of a 10× stock solution of 9 primer pairs (concentration of 0.05 μg/μl). This reaction was cycled 5, 8,10 or 35 cycles. Following round one amplification, the PCR products were diluted for use as templates in round two. In round two, 10 μl of PCR reaction were prepared by placing 9 μl of a master mixture containing buffer, MgCl₂, Taq polymerase and a primer pair specific for one gene into tubes containing 1 μl of each of the following dilutions of PCR product from the round one: undiluted, ⅕, 1/10, 1/50, 1/100, 1/1,000, 1/10,000, 1/100,000 and 1/1,000,000. These reactions were cycled 35 times. Primer pairs used in round two were selected from among the primer pairs used in round one. No additional cDNA or CT mixture was added into the PCR reaction in round two. For control uniplex START-PCR reactions, the mixture of cDNA and CT mixtures prepared for use in round one of the nine gene multiplex reactions was serially diluted prior to amplification: undiluted, ⅕, 1/10, 1/50, 1/100, 1/1,000, 1/10,000. These reactions were amplified in only one round of 35 cycles.

A 1 μl aliquot of each dilution was combined with an aliquot of a master mixture containing buffer, MgCl₂, Taq polymerase and a primer pair specific for one gene (0.05 μg/μl of each primer). Quantification of gene expression was determined.

FIGS. 4A-D show representative results of multiplex StaRT-PCR vs. uniplex StaRT-PCR reactions. For FIGS. 4A-D, following amplification, StaRT-PCR products were electrophoresed on 4% agarose gels. G.E.N.E. system 1a CT mix D and cDNA from A549 p85 were mixed together and amplified in uniplex and multiplex StaRT-PCR reactions.

FIG. 4A: Control uniplex reaction with β-actin primers. Lane 1, pGEM size marker; lane 2, PCR reaction contained undiluted cDNA in which β-actin NT in balance with 300,000 molecules of β-actin CT; lane 3, PCR reactions contained 1:5 diluted cDNA/CT mix; lane 4, 1:10 diluted cDNA/CT mix; lane 5, 1:50 diluted cDNA/CT mix; lane 6, 1:100 diluted cDNA/CT mix; lane 7, 1:1,000 diluted cDNA/CT mix; lane 8,1:10,000 diluted cDNA/CT mix.

FIG. 4B: PCR products from the second round multiplex StaRT-PCR reactions, each of which contained an aliquot of round one PCR product and β-actin primers. Lane 1, pGEM size marker; lane 2, 1/500th of the round one 10 μl PCR product (1 μl of a 1:50 dilution); lane 3, 1/1,000th round one PCR product; lane 4, 1/10,000th round one PCR product; lane 5, pGEM size marker, lane 6, 1/10,000th round one PCR product; lane 7, 1/100,000th round one PCR product; lane 8, 1/1,000,000th round one PCR product; lane 9, 1/10,000,000th round one PCR product.

FIG. 4C: Control reaction with catalase primers. Lane 1, pGEM size marker; lane 2, PCR reaction contained undiluted cDNA and CT mix, equivalent to 3,000 molecules of catalase CT; lane 3,1:5 diluted cDNA/CT mix; lane 4,1:10 diluted cDNA/CT mix; lane 5,1:50 diluted cDNA/CT mix; lane 6, 1:100 diluted cDNA/CT mix; lane 7, 1:1,000 diluted cDNA/CT mix; lane 8, 1:10,000 diluted cDNA/CT mix.

FIG. 4D: PCR products for the second round of multiplex StaRT-PCR. Reactions included an aliquot of round one PCR product and catalase primers. Lane 1, pGEM size marker; lane 2, 1/100th of the 10 μl round one PCR product (1 μl of a 1:10 dilution); lane 3, 1/500th round one PCR product; lane 4, 1/1,000th round one PCR product; lane 5, 1/10,000th round one PCR product; lane 6, 1/100,000th round one PCR product; lane 7, 1/1,000,000th round one PCR product; lane 8, 1/10,000,000th round one PCR product.

FIG. 5 is a graph showing the correlation of gene expression values obtained by either 96 gene Multiplex or Uniplex StaRT-PCR. Samples of cDNA derived from Stratagene Universal Human Reference RNA were combined with CT mix (mixes B, C, D, E and F from G.E.N.E. system 1 were used) and amplified either by uniplex StaRT-PCR or by 96 gene multiplex StaRT-PCR with primer pairs for all genes in G.E.N.E. system 1. Mean values are presented in FIG. 2 (Table 2) for the 93 genes that could be evaluated. Of these, 79 were measured by both uniplex and multiplex StaRT-PCR and could be compared. Gene expression values are presented as molecules of mRNA per 10⁶ β-actin mRNA molecules. Values obtained by uniplex StaRT-PCR are plotted along the X axis and values obtained by multiplex StaRT-PCR are plotted along the Y axis.

FIG. 6 is a table showing the expression measurement of putative carboplatin chemoresistant genes in primary non-small cell lung cancer (NSCLC).

FIG. 7 is a table showing a gene expression measurement in lung donor airway epithelial cells by multiplex StaRT-PCR.

FIG. 8 is a schematic diagram showing a software program useful in the system for quantitatively measuring the gene expression in small biological samples using the multiplex StaRT-PCR process.

### DESCRIPTION OF THE INVENTION

The use of the multiplex StaRT-PCR process to identify patterns of gene expression has many advantages. Because data are standardized, the data are readily comparable between samples and between laboratories. The numerical correlation of gene expression with clinically relevant phenotypes allows for the combination of gene expression values into indices that better define specific phenotypes. Compared to other methods of measuring gene expression, the multiplex StaRT-PCR process is rapid, inexpensive and sensitive. The presence of internal standards (CTs) in each reaction also allows for the quantitative multiplex StaRT-PCR process in which cDNA and CTs are amplified in two rounds. In addition, the amount of starting sample material required to measure expression is significantly less than the amount required by other methods. The standardized RT- PCR process (StaRT-PCR) allows rapid, reproducible, standardized, quantitative measurement of data for many genes simultaneously.

According to the method of the present invention, an internal standard competitive template (CT) is prepared for each gene, cloned to generate sufficient competitive templates for at least 10⁸, and preferably enough for >10⁹ assays and CTs for up to at least about 1000 genes are mixed together.

Competitive templates (CTs) were constructed essentially based on the method of Celi (Celi, F. S. et al., Nucleic Acids Res. 21, 1047 (1993)). Primers were initially designed using Primer 3.1 software to amplify from 200 to 800 bases of the coding region of targeted genes with an annealing temperature of 58° C. (tolerance of ±°1C.). This allowed all standardized analytical PCR reactions to be run under identical conditions and further allows for automation and high throughput applications, including microfluidic capillary gel electrophoresis. Before each CT was constructed, each primer pair was tested using reverse transcribed RNA (Research Genetics, Inc.) from a variety of tissues or individual cDNA clones known to represent the gene of interest. For primer pairs that failed (about 10% of the time) new ones were designed and the process repeated. For each gene, a CT primer (a fusion oligo of ˜40bp) then was prepared. The 3′ end of each fusion primer consisted of an ˜20 base sequence homologous to a region about 50-100 base 3′ to the reverse primer. The 5′ end was the 20 bp reverse primer. Competitive templates then were generated by running five 10 μl PCR reactions using the native forward primer and the CT primer. These PCR reactions were combined, electrophoresed on a 3% NuSieve gel in 1×TAE, and the band of correct size was cut from the gel and extracted using QiaQuick method (Qiagen, Valencia, Calif.). The purified PCR products were cloned into the PCR 2.1 vector using the TOPO TA cloning kits (Invitrogen, Carlsbad, Calif.) and were transformed into HS996 (a T1-phage resistant variant of DH10B).

After cloning, transformation, and plating on LB plates containing X-Gal, IPTG, and carbenicillin, three isolated white colonies were picked. Plasmid minipreps were made, EcoRI digestion was performed and the digests were electrophoresed on 3% SeaKem agarose. For those clones positive for an insert by EcoRI digestion, the sequence of the insert was determined by sequencing the same undigested plasmid preparation using vector specific primers. Only those clones that showed homology to the correct gene sequence and which had 100% match for the primer sequences were allowed to proceed to large-scale CT preparation and to be included in the standard mixes. Those that passed this quality control assessment then continued to the next steps. Plasmids from each quality clone then were prepared in quantities large enough (1.5 L) to allow for >1 billion assays (approximately 2.6 mg). The plasmids were purified from the resultant harvested cells using the Qiagen GigaPrep kit. Plasmid yields were assessed using the Hoeffer DyNAQuant 210 fluorometer.

An aliquot of each plasmid preparation was again sequenced at this step to assure quality. For each CT that passed all of the defined quality control steps we assessed the sensitivity of the preparation and primers by performing PCR reactions on serial dilutions and determining the limiting concentration that still yielded a PCR product. Only those preparations and primers that allow for detection of 60 molecules were continued for inclusion into standardized CT mixtures. Most of the assays that were developed had a sensitivity of 6 molecules or less.

Plasmids from quality assured preparations were mixed into CT mixtures representing either 24, or 96 genes. The concentration of the competitive templates in the 24 gene mixes were 4×10⁻⁹ M for β-actin CT, 4×10⁻¹⁰ M for GAPDH (CT1), 4×10⁻¹¹ M for GAPDH (CT2), and 4×10⁻⁸ M for each of the other CTs. The 24 gene CT mixes were linearized by NotI digestion prior to preparation of the working dilutions described below. Four 24-gene CT mixes were combined in equal amounts to yield 96-gene CT mixes at a top level concentration of 10⁻⁹ M for β-actin, 10⁻¹⁰ M GAPDH (CT1), 10⁻¹¹ M GAPDH (CT2), and 10⁻⁸ M for the other CTs. These top level mixes then were serially diluted with the 10⁻⁹ M β-actin, 10⁻¹⁰ M GAPDH (CT1), 10⁻¹¹ M GAPDH (CT2) mix, yielding six working standardized CT mixes (A-F) at concentrations of 10⁻¹² M for β-actin, 10⁻¹³ M for GAPDH CT1, 10⁻¹⁴ M for GAPDH2, and 10⁻¹¹(A), 10⁻¹²(B) 10⁻¹³(C) 10⁻¹⁴(D) 10¹⁵(E) 10⁻¹⁶ M (F) for the other CTs.

Each gene and reference, or housekeeping, gene is measured relative to respective CTs. Each target gene is then normalized to a reference gene to control for cDNA loaded into the reaction. Each gene expression measurement is reported as a numerical value that allows for direct inter-experiment comparison, for entry into a common databank, and for the combination of values into interactive gene expression indices. As long as the same mixture of internal standard CTs is used, direct comparisons may be made among samples within the same experiment, different experiments in the same laboratory, and potentially different experiments in different laboratories. For the experiments reported herein β-actin or GAPDH is the arbitrarily chosen reference, or housekeeping, gene in most StaRT-PCR studies. However, because in each multiplex StaRT-PCR process the experiment data are measured against a common mixture of internal standards, any measured gene or combination of genes (even all genes) can be used as the reference gene and the data easily re-calculated relative to that reference if so desired.

Within a cDNA sample, re-calculating relative to a new reference alters the value of each individual gene but does not alter the expression value of genes relative to each other.

In the multiplex StaRT-PCR method of the present invention cDNA and CTs are amplified in two rounds, which greatly increases the number of gene expression measurements obtainable from a small cDNA sample.

With the use of multiplex StaRT-PCR method, at least about 10,000 to at least about 100,000 gene expression measurements are obtained from the same amount of cDNA typically used to obtain one gene expression measurement using the Willey and Willey et al. '390, '606, and '978 StaRT-PCR processes. Since the same amount of cDNA is used in round one of the multiplex process as is used in the uniplex StaRT-PCR, rare transcripts are not diluted out and can still be detected with statistical significance.

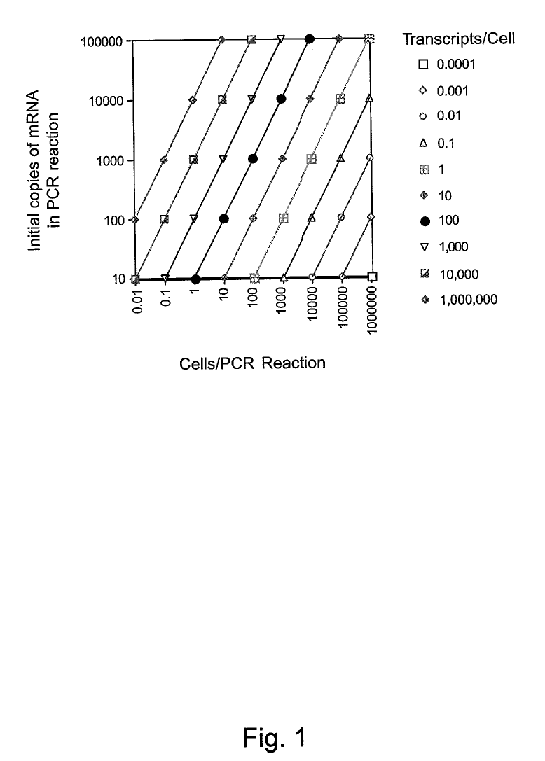

As demonstrated in FIG. 1, the amount of cDNA used in a PCR reaction has a direct relationship to the number of transcripts/cell that can be measured. It generally is assumed that RNA extraction is close to 100% whereas reverse transcription is 10% efficient. Thus, if a homogeneous population of cells is studied and each cell contains 10 copies of mRNA for a gene, 1 copy per cell will remain after reverse transcription. The statistical significance of measuring less than 10 copies of a transcript in a PCR is questionable. Thus, to detect 10 copies of a transcript, cDNA representing 10 cells must be present in the PCR reaction (FIG. 1). If a heterogeneous cell population is studied in which 1 cell out of 10 expresses a particular transcript, cDNA representing 1,000 cells must be present in the PCR reaction in order to detect 10 copies.

In the uniplex StaRT-PCR process, cDNA representing 100-1,000 cells is typically used to measure one gene in one PCR reaction. Using this amount, according to FIG. 1, it is possible to detect transcripts that are expressed at 0.1-1 copy per cell (or 1-10 copies per 10 cells) with statistical significance. The same amount of cDNA is used in the first round of multiplex StaRT-PCR process. Since this cDNA is co-amplified with CTs for each gene to be measured and since the relationship of endogenous cDNA to CT remains constant after PCR, the PCR product from round one can be diluted and reamplified again in a second round with primers specific to one gene without significantly changing the numerical values obtained relative to those obtained with uniplex StaRT-PCR. In this manner, sufficient PCR product can be generated to detect and measure gene expression for many genes without using additional cDNA and without significantly changing the numerical values obtained with the traditional StaRT-PCR process.

Microfluidic CE technology (T. S. Kanigan et al., in Advances in Nucleic Acid and Protein Analyses, Manipulation, and Sequencing, P. A. Limbach, J. C. Owicki, R. Raghavachari, W. Tan, Eds. Proc. SPIE 3926: 172, 2000) allows measurement of gene expression in very small volumes. However, as discussed above, there is a minimum amount of cDNA that can be used and still achieve a statistically significant measurement. In over 50,000 StaRT-PCR gene expression measurements involving over 200 different genes and over 100 cell line, and tissue samples, there is a stochastic distribution of expression among genes with the mean approximately 2 logs lower than β-actin. A typical 1 μl cDNA sample representing about 1,000 bronchial epithelial cells is in balance with 6×10⁵ β-actin CT. Genes expressed at the mean level (100-fold lower than β-actin), would be in balance with about 6,000 CT molecules. However, a small number of genes (but often very important functionally) are expressed 10,000-fold lower than β-actin, and for such genes there would be 60 molecules represented in this sample. If one were to reduce the volume of this PCR reaction 100-fold from 10 μl to 100 nanoliters, genes expressed 10,000-fold lower than β-actin would be represented by 0.6 molecules or fewer which, due to stochastic considerations, would be difficult to quantify with acceptable confidence. In contrast, with the multiplex StaRT-PCR process, 10 nanoliters of a 10 μl round one PCR product may be used in the round two PCR reaction volume of 100 nanoliters. Because more than 1,000,000-fold amplification is routinely achieved in the round one reaction, 10 nanoliters of the 10 μl round one reaction will contain ample native and competitive templates to be measured with statistical confidence in the round two reaction.

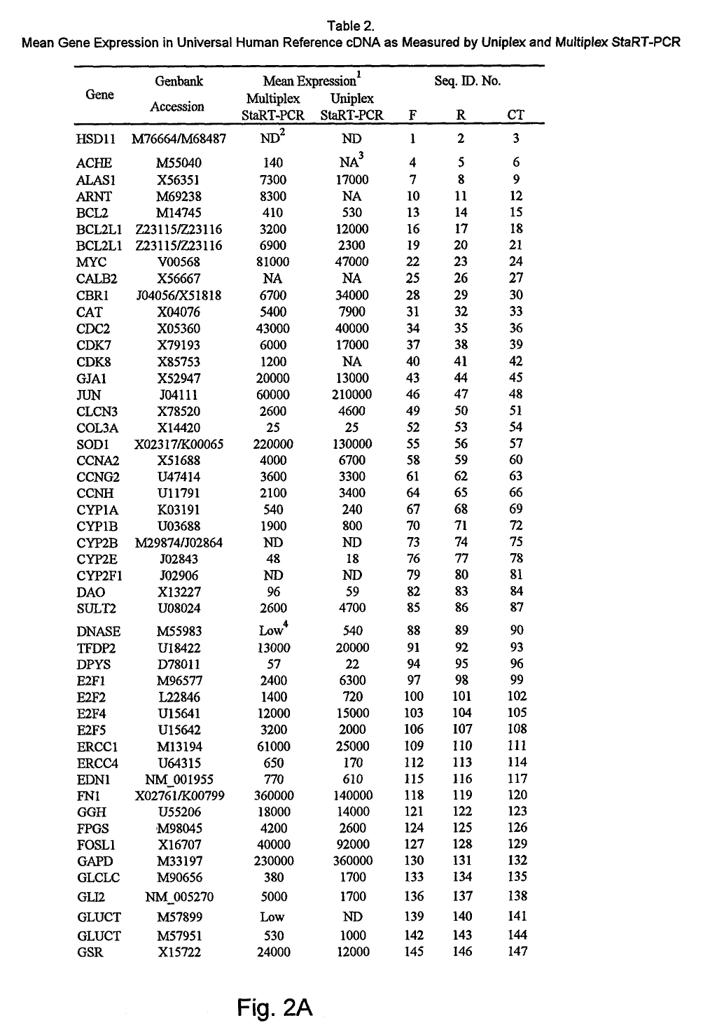

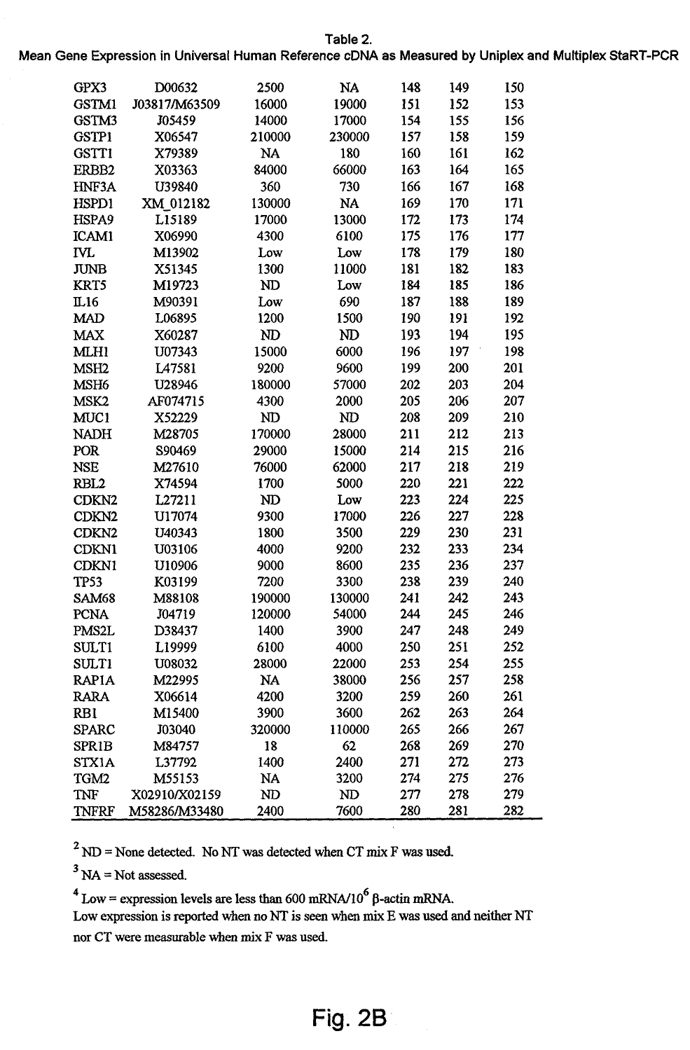

The table in FIG. 2 shows the primer sequence and position for several genes.

Automation of StaRT-PCR by combining multiblock thermal cyclers with a slightly modified CE system allows over 4,000 gene expression assays/24 hours. Further, by designing primers that amplify native template (NT) and competitive template (CT) product sizes distributed between 200 and 800 bp, it is possible to quantify more than 100 genes on a single CE channel. Thus, 96-channel CE devices may be converted to automated, high throughput (>300,000 standardized gene expression assays/24 hours) devices with little difficulty.

The most efficient approach for StaRT-PCR analysis, in terms of cDNA consumption and cost is to: 1) dilute the cDNA sample to be tested so that 1 μl is in balance with 600,000 molecules of β-actin CT (1 μl of CT mix); 2) use 1 μl of balanced cDNA in round one of two-step StaRT-PCR process with each of the six (A-F) CT mixes; 3) use 10 nanoliters of the round one StaRT-PCR product in parallel 100 nanoliter volume round two reactions to measure expression of all 96 System 1 genes using Mix D (which contains CTs at a concentration that will be in balance with the majority of genes); and, 4) repeat StaRT-PCR for the genes that are not in balance with Mix D using the appropriate mix.

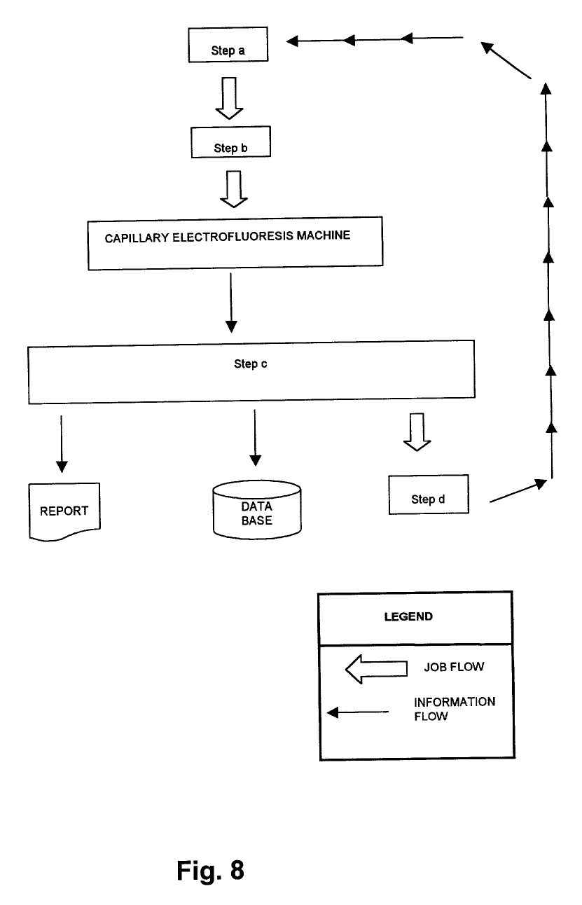

A software program that selects, based on the NT/CT ratio for each gene, the appropriate CT mix to use in repeat experiments, is useful and is also within the scope of the present invention. FIG. 8 is a schematic diagram that shows, in combination, in a system for quantitatively measuring the gene expression a plurality of target genes of interest of the method, a software program which performs the steps of: a) determining a desired concentration of CT reagents to be used when conducting a competitive form of reverse transcription-polymerase chain reaction (RT-PCR) of samples the target genes; b) selecting and causing to be dispensed desired at least one reagent into a plurality of reaction chambers in which the RT-PCR is to be conducted; and, sending to a suitable device for identifying and/or labeling, for example, by flowing to a capillary electrofluoresis (CE) machine and sometimes ending the process there. In certain embodiments, information from the CE machine goes to step c) for analyzing quantitative data about the resultant product to quantitatively measure the expression of the target genes. The information from step c) can be provided in a “Report”, sent to a “Database” and/or sent to step d) which reiterates the process for further analysis of the data.

A computer program and product for quantitatively measuring gene expression of target genes of interest through a two-step quantitative RT-PCR process includes a computer readable medium; and, instructions, stored on the computer readable medium, for quantitatively measuring gene expression. The instructions preferably include the steps recited above.

The computer program and product can further include instructions for including dispensing PCR reaction mixtures into high density cDNA and/or oligonucleotide arrays to measure PCR products following quantitative RT-PCR.

The computer program and product can further include the instructions for fluorescently labeling the oligonucleotide hybridizing the sense strand and/or anti-sense strand of each cDNA being amplified.

The computer program and product can further include instructions for labeling with a fluorescent dye one or more of the dNTP's within the oligonucleotide in the PCR reaction.

The computer program and product can further include instructions where the expression of the target genes are quantified by comparing the fluorescent intensities of the arrays for the native and CT for the housekeeping genes and targets genes.

The present invention also includes a computer implemented method for quantitatively measuring gene expression of a plurality of target genes of interest using the multiplex RT-PCR process using the steps described above.

FIG. 8, shows a diagrammatic flowchart for the present invention for optimizing the quantitative measurement of the genes using a further step (d) for further analyzing the data received to determine whether the calculated ratio is within a desired range (for example, within a 10-fold ratio). If the calculated ratio is not within the desired range, a new desired concentration of CT reagents (i.e., different from the original concentration selected to step (a)) is chosen and the steps (b)-(c) are repeated with the new concentration of CT reagents.

While gene expression may be measured at the mRNA, protein, or functional level, measurement at the mRNA level is particularly suitable for development of a common language for gene expression. This is because mRNA expression is regulated primarily by the number of transcripts available for translation. In contrast, at the protein level, copy number often is less important than modifications including phosphorylation, dimerization, and/or proteolytic cleavage. Because mRNA expression is related primarily to copy number, one is able to develop an internal standard for each gene and also to establish a common unit for gene expression measurement. Although reverse transcription efficiency is variable, the representation of one gene to another in the resultant cDNA is not affected, thus target gene cDNA copies/10⁶ β-actin cDNA copies will be equivalent to target gene mRNA/10⁶ β-actin mRNA.

Gene expression is measured in the experiments herein in reference to β-actin mRNA. However, if it is determined that another gene, e.g. GAPDH or any other gene measured, or even all genes measured, is more stable across samples, the data may be re-calculated to that reference gene without altering the relative expression value within a sample.

When gene expression data are re-calculated using GAPDH or any other gene as the reference gene, the relative expression among genes remains the same. Conversion from mRNA/10⁶ β-actin molecules reference to mRNA/10⁶ GAPDH molecules reference is done simply by multiplying each expression value by 10⁶/(GAPDH mRNA/10⁶ β-actin molecules). When this done, the relative expression of genes within the same sample does not change. Thus, the expression value ratio between two genes within a sample would be the same with GAPDH, β-actin, or a combination of genes as the reference. The reason for this is that expression measurement of each of the genes is linked through the CT mixture and as long as the same CT mixture is used, the concentration ratio from one CT to another remains the same. In the case of gene expression indices, the difference in value obtained after converting from one reference gene to another is dependent on how many genes are in the numerator and how many are in the denominator. Each gene in a gene expression index must be converted to the new reference prior to calculation of the index. If there are equal numbers of genes in the numerator and denominator, the conversion to a new reference has no effect on the relative index value between samples. However, if there are non-equal numbers of genes in the numerator and denominator, the relative index value between samples will change in accordance with any difference in the relative reference gene value between samples.

The effect of a reference gene that varies in expression from one sample to another is neutralized in interactive gene expression indices that are balanced (i.e. equal numbers of expression values in the numerator and denominator). Because of this, and because interactive gene expression indices correlate better with phenotype than the expression of individual genes, it is desirable to seek balanced interactive gene expression indices that correlate with the phenotype of interest.

It is possible to identify primers that will PCR amplify both human and mouse for about 30% of genes for which reagents are currently available. Primers are being developed to obtain even wider cross-species application. Thus, the multiplex StaRT-PCR reagents provide a common language for gene expression across species.

The data presented below in the examples confirm that the multiplex StaRT-PCR process allows reliable inter-laboratory comparison of gene expression data. The multiplex StaRT-PCR process allows replicate measurement of many genes in small samples. The multiplex StaRT-PCR method is well suited to high throughput automation and miniaturization. With the level of sensitivity and reproducibility, as presented herein, the multiplex StaRT-PCR process promotes the development of a meaningful gene expression database and serves as a common language for gene expression.

### EXAMPLE I

Materials and Methods

Reagents

10×PCR buffer for the Rapidcycler (500 mM Tris, pH 8.3, 2.5 mg/μl BSA, 30 mM MgCl₂ was obtained from Idaho Technology, Inc. (Idaho Falls, Id.). Thermo 10× buffer (500 mM KCl, 100 mM Tris-HCl, pH 9.0, 1.0% Triton X-100), taq polymerase (5 U/μl), oligo dT primers, RNasin (25 U/μl), pGEM size marker, and dNTPs were obtained from Promega (Madison, Wis.). M-MLV reverse transcriptase (200 U/μl) and 5× first strand buffer (250 mM Tris-HCl, pH 8.3, 375 mM KCl, 15 mM MgCl₂, 50 mM DTT) were obtained from GibcoBRL (Gaithersburg, Md.). NuSieve and SeaKem LE agarose were obtained from FMC BioProducts (Rockland, Me.). TriReagent was obtained from Molecular Research Center (Cincinnati, Ohio). RNase-free water was obtained from Research Genetics (Huntsville, Ala.). DNA 7500 Assay kit containing dye, matrix and standards was obtained from Agilent Technologies (Palo Alto, Calif.). The lung adenocarcinoma cell line, A549, was purchased from American Type Culture Collection (Rockville, Md.). RPMI-1640 cell culture medium was obtained from Sigma (St. Louis, Mo.). Universal Human Reference RNA was obtained from Stratagene (La Jolla, Calif.). Oligonucleotide primers were custom synthesized by Biosource International (Menlo Park, Calif.). G.E.N.E. system 1 and system 1a gene expression kits were kindly provided by Gene Express National Enterprises, Inc. (Huntsville, Ala.). All other chemicals and reagents were molecular biology grade.

RNA Extraction and Reverse Transcription

Total RNA from cells grown in monolayer was extracted according to the TriReagent Manufacturer Protocol. Universal Human Reference RNA was precipitated according to the manufacturer protocol. Approximately 1 μg total RNA was reverse transcribed using M-MLV reverse transcriptase and an oligo dT primer.

Uniplex StaRT-PCR

StaRT-PCR was performed using previously published protocols (Willey, J. C. et al., Am. J. Respir. Cell Mol. Biol. 19: 6-17,1998; Gene Express System1 Instruction Manual, Gene Express National Enterprises, Inc. www.genexnat.com 2000) with G.E.N.E. system 1 or system 1a gene expression kit (Gene Express National Enterprises, Inc.). Briefly, a master mixture containing buffer, MgCl₂, dNTPs, cDNA, competitive template (CT) mixture from G.E.N.E. system 1 or system 1 a kit and taq polymerase was prepared and aliquotted into tubes containing gene-specific primers and cycled either in a Rapidcycler (Idaho Technology, Inc.) or Primus HT Multiblock thermal cycler (MWG-BIOTECH, Inc., High Point, N.C.) for 35 cycles. In each protocol the denaturation temperature was 94° C., the annealing temperature was 58° C., and the elongation temperature was 72° C. For the Rapidcyler, the denaturation time was 5 seconds, the annealing time was 10 seconds, the elongation time was 15 seconds and the slope was 9.9. For the Primus HT Multiblock, the denaturation, annealing and elongation times were each 1 minute, the lid temperature was 110° C. and the lid pressure was 150 Newtons. PCR products were evaluated on an agarose gel or in the Agilent 2100 Bioanalyzer (Agilent Technologies, Inc.) as described below.

Multiplex StaRT-PCR Amplification of Nine Genes

Each multiplex StaRT-PCR reaction was amplified in two rounds. In the first round of the multiplex StaRT-PCR process, one reaction was set up containing buffer, MgCl₂, dNTPs, a previously prepared mixture of cDNA and CT mixture (1:1 cDNA from A549 p85 and one of the CT mixes from G.E.N.E. system 1a), taq polymerase and primer pairs for 9 genes. This reaction was cycled 5, 8, 10 or 35 cycles. The concentration of each primer in the primer mix was 0.05 μg/μl. Following this amplification, this PCR product was diluted with water for use as a template in round two.

In round two, a master mixture containing buffer, MgCl₂, taq polymerase and a primer pair specific for one gene was aliquotted into tubes containing 1 μl of each of the following dilutions of PCR product from the first round: undiluted, ⅕, 1/10, 1/50, 1/100, 1/1,000, 1/10,000, 1/100,000 and 1/1,000,000. These reactions were cycled 35 times and detected on an agarose gel or in the Agilent 2100 Bioanalyzer as described below. Primer pairs used in this round were selected from among the primer pairs used in round one. No additional cDNA or CT mixture was added into the PCR reaction in round two.

For control uniplex StaRT-PCR reactions, the mixture of cDNA and CT mixture prepared for use in round one of the nine gene multiplex reactions was serially diluted prior to amplification: undiluted, ⅕, 1/10, 1/50, 1/100, 1/1,000, 1/10,000. A 1 μl aliquot of each dilution was combined with an aliquot of a master mixture containing buffer, MgCl₂, Taq polymerase and a primer pair specific for one gene (0.05 μg/μl of each primer). These reactions were amplified with only one round of 35 cycles.

Multiplex StaRT-PCR Amplification of Ninety-Six Genes

Samples of cDNA derived from Stratagene Universal Human Reference RNA and CT mixes from G.E.N.E. system 1 (which contain CTs for 96 genes) were used in these experiments. A solution containing primers for each of the 96 genes represented by CTs in G.E.N.E. system 1 was included in the first round reactions. This 96 gene primer mix was diluted so that the concentration of each primer was 0.005 μg/μl. Every round one reaction was cycled 35 times. Round one PCR products then were diluted 100-fold (1 μl of round one product into 99 μl water). One microliter of diluted round one PCR product was used in each round two reaction along with primers for a single gene selected from among those amplified in round one, and cycled 35 times.

Control uniplex reactions were conducted using samples of cDNA derived from Stratagene Universal Human Reference RNA and CT mixes from G.E.N.E., system 1 as described above. For these experiments, no dilution of the cDNA or CT mix was done prior to amplification.

Electrophoresis and Quantitation

Agarose Gel Electrophoresis:

Following amplification, PCR products were loaded directly on to 4% agarose gels (3:1 NuSieve:SeaKem) containing 0.5 μg/ml ethidium bromide. Gels were electrophoresed for approximately one hour at 225V. Electrophoresis buffer was cooled and recirculated during electrophoresis. Gels were visualized with a Foto/Eclipse image analysis system (Fotodyne, Hartland, Wis.). Digital images were saved on a Power Mac 7100/66 computer and Collage software (Fotodyne) was employed for densitometric analysis (or were analyzed using Agilent 2100 Bioanalyzer (as discussed below)).

Quantification of gene expression was determined. First, the native template (NT)/CT ratio of a housekeeping gene, β-actin, and the NT/CT ratios for each target gene were calculated. Because the initial concentration of CT added into the PCR reaction was known, the initial NT concentration could be determined. Since each NT/CT ratio was based on ethidium bromide staining of the PCR products and this staining is affected by both the number of molecules present and the length of the molecules in base pairs, NTs were arbitrarily corrected to the size of the CT product prior to taking the NT/CT ratio. Heterodimers (HD), when measurable, were corrected to the size of the CT and divided by two. One half of the HD value was added to the NT and one half was added to the CT prior to taking the NT/CT ratio since one strand of the HD comes from the NT and the other comes from the CT. Second, the calculated number of target gene NT molecules was divided by the calculated number of β-actin NT molecules to correct for loading differences.

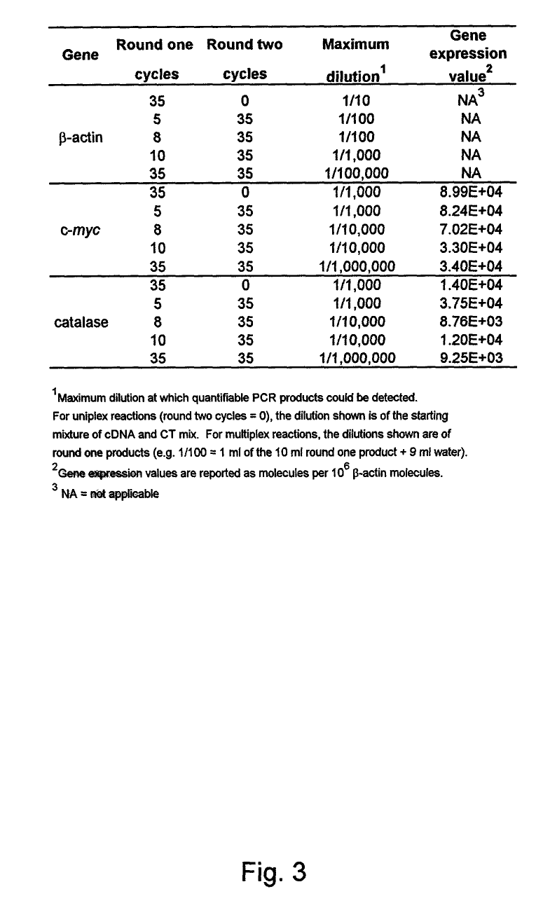

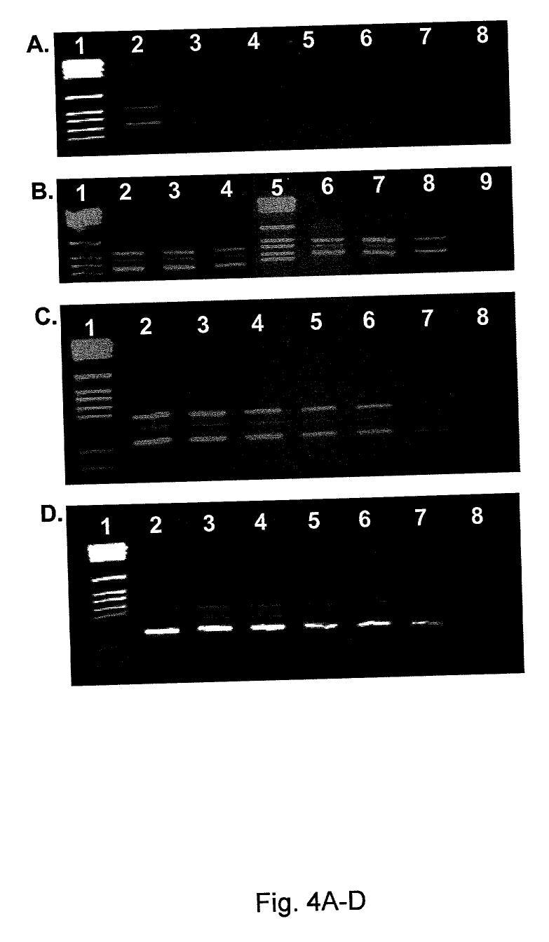

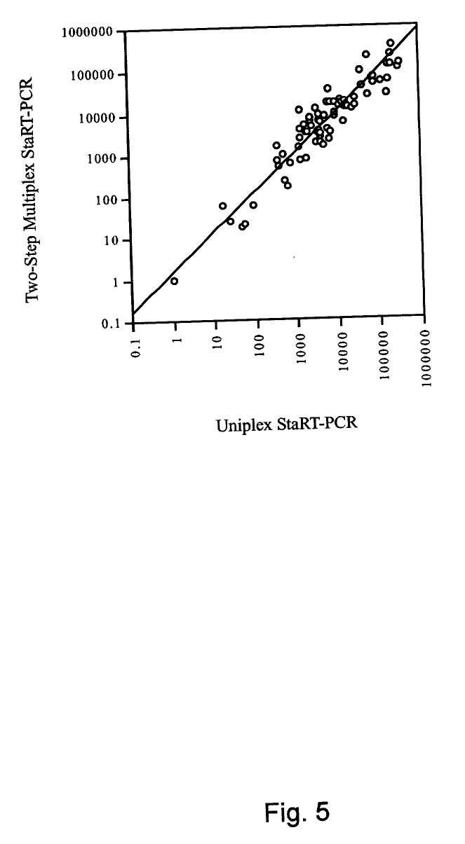

For multiplex StaRT-PCR, target genes detected under each condition (varying dilution and/or round one cycle number) were measured against β-actin detected under the same condition. For example, round one of the nine gene multiplex reaction contained primers for nine genes including both β-actin and c-myc. A 1/100,000 dilution of the PCR reaction from round one was made and used in round two. An aliquot of this dilution was used in round two to amplify both β-actin and c-myc. Under these conditions, c-myc was measured as 3.40×10⁴ molecules/10⁶ β-actin molecules when cycled 35 times in round one and 35 times in round two (FIG. 3).

Agilent 2100 Bioanalyzer Microcapillary Electrophoresis:

Following amplification, 1 μl of each 10 μl PCR reaction was loaded into a well of a chip prepared according to the manufacturer's protocol for the DNA 7500 Assay. Briefly, 9 μl gel-dye matrix was loaded into the chip in one well and the chips were pressurized for 30 seconds. Two additional wells were filled with gel-dye matrix and the remaining wells each were loaded with 5 μl of molecular weight marker. One microliter of DNA ladder was loaded into a ladder well and 1 μl of PCR product was loaded into each sample well. The chip was vortexed and placed into the Agilent 2100 Bioanalyzer. The DNA 7500 Assay program was run which applies a current sequentially to each sample to separate products. DNA was detected by fluorescence of the intercalating dye in the gel-dye matrix. NT/CT ratios were calculated from the area under the curve for each PCR product and a size correction was made since, as with ethidium bromide stained agarose gel electrophoresis, an intercalating dye was used to detect DNA.

Statistical Analysis:

All statistical analyses were conducted using SPSS version 9.0 for Windows. A two-tailed Pearson Correlation test was conducted on logarithmically transformed data to compare gene expression values obtained by uniplex StaRT-PCR with those obtained by multiplex StaRT-PCR. The correlation was considered statistically significant if the p value was less than 0.05.

Results

Multiplex StaRT-PCR Amplification of Nine Genes

After 35 cycles of amplification in round one with primer pairs for nine genes, aliquots of the PCR products were diluted and amplified with primers for one of the nine genes. Bright, distinct bands were observed for each gene (FIG. 1). Thus, the same amount of cDNA and CT mix that is used in a typical uniplex StaRT-PCR reaction to measure one gene in one round of amplification was used to obtain nine gene expression measurements in multiplex StaRT-PCR.

Further, the round one PCR product can be diluted as much as 1,000,000-fold for catalase or c-myc (1 00,000-fold for β-actin) and still be quantified following amplification with primer pairs for one gene in round two (FIGS. 1 and 3). In contrast, when the cDNA and CT mix used in round one was diluted more than 1,000-fold prior to amplification (100-fold or more for β-actin) and then amplified with a single primer pair for any one of these genes in a single round of 35 cycles, no detectable product was observed.

Increasing the number of cycles used in round one increased the amount the PCR product that could be diluted prior to round two and still be detectable after round two amplification. Therefore, more gene expression measurements can be made on a sample when it is amplified using multiplex StaRT-PCR with 35 cycles used in each round than when fewer cycles (5, 8 and 10 cycles) are used in round one or when uniplex StaRT-PCR is used. Details for each gene and each condition are shown in FIG. 3. Representative gels of control uniplex reactions and multiplex reactions are shown in FIG. 1.

Multiplex StaRT-PCR Amplification of Ninety-Six Genes

Gene expression values obtained by uniplex and 96 gene multiplex StaRT-PCR of the cDNA derived from Stratagene Universal Human Reference RNA are shown in FIG. 2. Although 96 primer pairs were included in the multiplex reactions, gene expression values for only 93 genes are reported because 1) each gene expression value is reported as molecules of target gene/10⁶ molecules of β-actin so β-actin values are not reported, 2) although two sets of reagents to measure GAPD gene expression (GAPD CT1 and GAPD CT2) are included in the G.E.N.E. system 1 kit, only GAPD CT1 was measured in this sample and, 3) reagents for one gene, BAX alpha, provided in the kit did not pass quality control testing done by G.E.N.E., Inc. so this gene was not assessed in this study. Bivariate analysis of uniplex and multiplex StaRT-PCR gene expression values revealed a highly significant (p=0.001) positive correlation (r=0.993) (FIG. 3).

### EXAMPLE II

The multiplex StaRT-PCR method of the present invention allows investigators to study more genes and to obtain more replicate data from small amounts of cDNA that are available from biopsies, micro-dissected tissues or sorted cell populations.

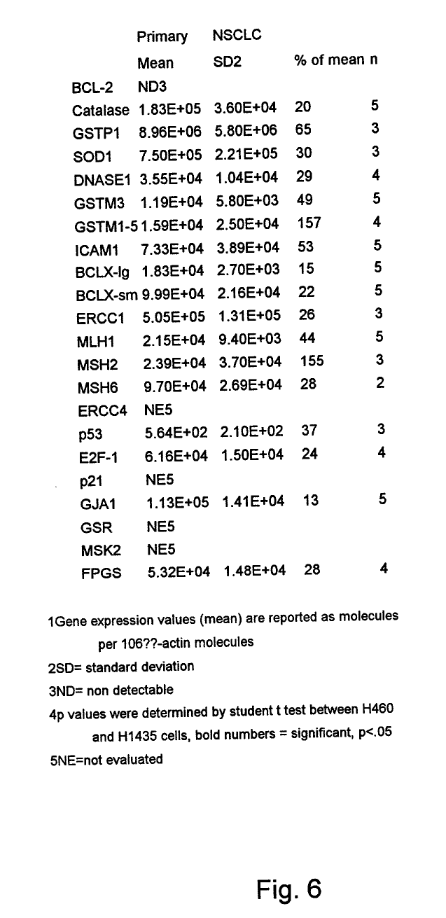

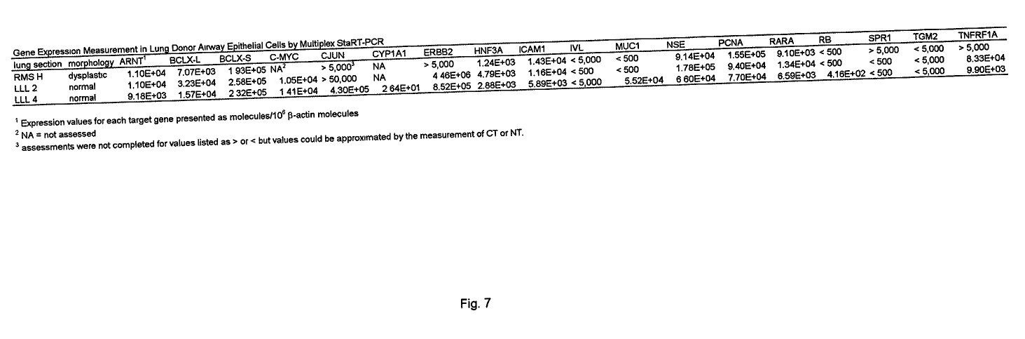

FIGS. 6 and 7 are tables of data that show that the method of the present invention as applied to small primary tissue cells successfully. FIG. 6 shows fine needle aspiration data collected from a non-small cell long-cancer (NSCLC). The gene measures are listed and all data was measured using the CT mixtures from the GENE System 1 by 18 multiplex PCR.

FIG. 7 shows the data collected from a lung donor who had no disease of the lung. The gene expression was also collected using 96 gene multiplex PCR with the CT mixes from the GENE System 1.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. A method for direct comparison of numerical gene expression values between samples of genes using reverse transcription-polymerase chain reaction, comprising:
i) combining cDNA, a competitive template mixture, and primer pairs for a plurality of genes with at least one suitable buffer and at least one suitable enzyme to form a first mixture and allowing the first mixture to be amplified for a predetermined number of cycles to form PCR products;
ii) mixing a predetermined amount of the PCR products from with at least one suitable buffer, at least one enzyme, and one primer pair specific for each of the genes to form a second mixture and allowing the second mixture to be amplified an additional predetermined number of cycles.

2. The method of claim 1, in which the PCR product in (i) is amplified for between about 3 to about 40 cycles.

3. The method of claim 1, in which the PCR product in (i) is amplified for 5, 8, 10 or 35 cycles.

4. The method of claim 1, in which the PCR product in (i) is amplified for about 35 cycles.

5. The method claim 1, in which the PCR products from (i) are diluted as much as 100,000 fold.

6. The method of claim 1, in which at least about 100,000 gene expression measurements are obtained from the (i) PCR product.

7. The method of claim 1, in which the primer pairs are diluted to about 0.05 to about 0.005 μg/μl when the number of genes to be compared increases.

8. The method of claim 1, in which the numerical gene expression value is correlated with clinically relevant phenotypes which allows a combination of the gene expression values into at least one index that defines at least one specific phenotype.

9. The method if claim 1, in which an internal standard competitive template is prepared for each gene and is cloned to generate competitive templates for at least about 10¹⁰ assays.

10. The method of claim 9, in which competitive templates for up to about 1000 genes are mixed together.

11. The method of claim 9, in which the assays have a sensitivity of about 6 molecules or less.

12. The method of claim 1, in which the competitive template (CT) mixture comprises at least one competitive template (CT) reference, or housekeeping, gene, and at least one target gene.

13. The method of claim 12, in which the CT mixture comprises a CT for at least one reference gene and a CT for at least one target gene.

14. The method of claim 12, in which the CT mixture comprises a CT for at least one reference gene and a combination of CTs for multiple target genes.

15. The method of claim 12, in which the at least one reference gene comprises β-actin.

16. The method of claim 12, in which the at least one reference gene comprises GAPDH.

17. The method of claim 1, in which the gene expression is quantified by calculating: i) a ratio of native template (NT) to competitive template (CT) for a reference gene; ii) ratios of NT/CT for each target gene; and (iii) a ratio of the target gene NT/CT ratio(ii) relative to the housekeeping NT/CT ratio(i).

18. The method of claim 1 which further includes use of high density oligonucleotide or cDNA arrays to measure PCR products following quantitative RT-PCR.

19. The method of claim 18, in which the oligonucleotide or cDNA hybridizing to the sense strand or reverse strand of each cDNA being amplified is fluorescently labeled.

20. The method of claim 18, in which one or more of the dNTP's within the oligonucleotide or cDNA in the PCR reaction is labeled with a fluorescent dye.

21. The method of claim 18, wherein the high density oligonucleotide arrays have the following properties: for each gene, two oligonucleotide arrays are prepared; i) one locus on an array has attached to it oligonucleotides that are homologous to, and will bind to, sequences unique to the native template for the gene that was PCR-amplified; and, ii) another locus on an array has attached to it oligonucleotides that are homologous to, and will bind to, sequences that span the juncture between the 5′ end of the competitive template, and the truncated, mis-aligned 3′ end of the competitive template.

22. The method of claim 18, in which the expression of the target genes are quantified by comparing the fluorescent intensities of the arrays for the native and CT for the housekeeping genes and targets genes.

23. In combination in a system for quantitatively measuring gene expression a plurality of target genes of interest of the method of claim 1, comprising a software program which performs the steps of:
a) determining a desired concentration of CT reagents to be used when conducting a form of reverse transcription-polymerase chain reaction (RT-PCR) of samples the target genes; and,
b) selecting and causing to be dispensed at least one desired reagent into a plurality of reaction chambers in which the RT-PCR is to be conducted; and suitably identifying the products of the RT-PCR process.

24. The method of claim 23, in which the identified RT-PCR products undergo a further step c) of analyzing quantitative data about the resultant product to quantitatively measure the expression of the target genes.

25. The system of claim 24, which further includes a step (d) of providing information in order to select and cause to be dispensed the desired reagents in order to optimize the process of quantitatively measuring gene expression, whereby if a desired ratio of target gene NT/CT ratio to housekeeping gene NT/CT ratio is not within a desired range, a second desired concentration is determined and the steps of (a) to (c) are repeated.

26. A computer program product for quantitatively measuring gene expression of target genes of interest through a quantitative RT-PCR process, the computer program product comprising:
a computer readable medium; and
instructions, stored on the computer readable medium, for quantitatively measuring gene expression, the instructions comprising:
a) automatically determining a desired concentration of CT reagents to be used when conducting a form of reverse transcription-polymerase chain reaction (RT-PCR) of samples the target genes;
b) selecting and causing to be dispensed desired reagents into a plurality of-reaction chambers in which to conduct the RT-PCR; and, optionally,
c) analyzing quantitative data about the resultant product to quantitatively measure the expression of the target genes.

27. The software program product of claim 26, wherein the instructions further comprise step (d):
of providing information in order to select and cause to be dispensed the desired reagents in order to optimize the process of quantitatively measuring gene expression, whereby if a desired ratio of target gene NT/CT ratio to housekeeping gene NT/CT ratio is not within a desired range, a second desired concentration is determined and the steps of (a) to (c) are repeated.

28. The computer program product of claim 26, where the instructions further comprise including dispensing PCR reaction mixtures into high density cDNA or oligonucleotide arrays to measure PCR products following quantitative RT-PCR.

29. The computer program product of claim 28, where the instructions further comprise fluorescently labeling the oligonucleotide or cDNA hybridizing the sense strand and/or anti-sense strand of each cDNA being amplified.

30. The computer program product of claim 26, where the instructions further comprise labeling with a fluorescent dye one or more of the dNTP's within the oligonucleotide in the PCR reaction.

31. The computer program product of claim 30, wherein the expression of the target genes are quantified by comparing the fluorescent intensities of the arrays for the native and CT for the housekeeping genes and targets genes.

32. A computer implemented method for quantitatively measuring gene expression a plurality of target genes of interest using a RT-PCR process, the method comprising:
a) determining a desired concentration of CT reagents to be used when conducting a competitive form of reverse transcription-polymerase chain reaction (RT-PCR) of samples the target genes;
b) selecting and causing to be dispensed desired reagents into a plurality of reaction chambers in which to conduct the RT-PCR; and, optionally,
c) analyzing quantitative data about the resultant product to quantitatively measure the expression of the target genes.

34. The method of claim 32, further comprising a step (d):
of providing information in order to select and cause to be dispensed the desired reagents in order to optimize the process of quantitatively measuring gene expression, whereby if a desired ratio of target gene NT/CT ratio to housekeeping gene NT/CT ratio is not within a desired range, a second desired concentration is determined and the steps of (a) to (c) are repeated.
